SEQUENCE LISTING (18 sequences)

SEQ ID NO: 1 - protein, 387 aa, synthetic construct
MMKYLLVFAIIATRIPVLLVIGSEDNRVPGDKKLTKEGAAALCKMKHLADKVAKERSQELKDRTQNFAGYIEFELYRIDYWLEKLNGPKGRKDGYAKLSDSDIEKVKEIFNKAKDGITKQLPEAKKAGEEAGKLHTEVKKAAENARGQDLDDDTAKSTGLYRVLNWYCITKEERHNATPNCDGIQFRKHYLSVNRSAIDCSSTSYEENYDWSANALQVALNSWEDVKPKKLESAGSDKNCNIGQSSESHPCTMTEEWQTPYKETVEKLRELEDAYQRGKKAHDAMLGYANTAYAVNTKVEQEKPLTEVIAAAKEAGKKGAKIIIPAAAPATPTNSTKNDDSAPTEHVDRGIATNETQVEVGIDADFDSLLDATEAAEVTRRHQRTAM

SEQ ID NO: 2 - protein, 28 aa, synthetic construct
YIEFELYRIDYWLEKLNGPKGRKDGYAK

SEQ ID NO: 3 - protein, 4 aa, synthetic construct
DWSA

SEQ ID NO: 4 - protein, 13 aa, synthetic construct
NTAYAVNTKVEQE

SEQ ID NO: 5 - protein, 346 aa, synthetic construct
LLVIGSEDNRVPGDKKLTKEGAAALCKMKHLADKVAKERSQELKDRTQNFAGYIEFELYRIDYWLEKLNGPKGRKDGYAKLSDSDIEKVKEIFNKAKDGITKQLPEAKKAGEEAGKLHTEVKKAAENARGQDLDDDTAKSTGLYRVLNWYCITKEERHNATPNCDGIQFRKHYLSVNRSAIDCSSTSYEENYDWSANALQVALNSWEDVKPKKLESAGSDKNCNIGQSSESHPCTMTEEWQTPYKETVEKLRELEDAYQRGKKAHDAMLGYANTAYAVNTKVEQEKPLTEVIAAAKEAGKKGAKIIIPAAAPATPTNSTKNDDSAPTEHVDRGIATNETQVEVGID

SEQ ID NO: 6 - protein, 365 aa, synthetic construct
MMKYLLVFAIIATRIPVLLVIGSEDNRVPGDKKLTKEGAAALCKMKHLADKVAKERSQELKDRTQNFAGYIEFELYRIDYWLEKLNGPKGRKDGYAKLSDSDIEKVKEIFNKAKDGITKQLPEAKKAGEEAGKLHTEVKKAAENARGQDLDDDTAKSTGLYRVLNWYCITKEERHNATPNCDGIQFRKHYLSVNRSAIDCSSTSYEENYDWSANALQVALNSWEDVKPKKLESAGSDKNCNIGQSSESHPCTMTEEWQTPYKETVEKLRELEDAYQRGKKAHDAMLGYANTAYAVNTKVEQEKPLTEVIAAAKEAGKKGAKIIIPAAAPATPTNSTKNDDSAPTEHVDRGIATNETQVEVGIDAD

SEQ ID NO: 7 - protein, 286 aa, synthetic construct
SEDNRVPGDKKLTKEGAAALCKMKHLADKVAKERSQELKDRTQNFAGYIEFELYRIDYWLEKLNGPKGRKDGYAKLSDSDIEKVKEIFNKAKDGITKQLPEAKKAGEEAGKLHTEVKKAAENARGQDLDDDTAKSTGLYRVLNWYCITKEERHNATPNCDGIQFRKHYLSVNRSAIDCSSTSYEENYDWSANALQVALNSWEDVKPKKLESAGSDKNCNIGQSSESHPCTMTEEWQTPYKETVEKLRELEDAYQRGKKAHDAMLGYANTAYAVNTKVEQEKPLTEV

SEQ ID NO: 8 - protein, 234 aa, synthetic construct
GYIEFELYRIDYWLEKLNGPKGRKDGYAKLSDSDIEKVKEIFNKAKDGITKQLPEAKKAGEEAGKLHTEVKKAAENARGQDLDDDTAKSTGLYRVLNWYCITKEERHNATPNCDGIQFRKHYLSVNRSAIDCSSTSYEENYDWSANALQVALNSWEDVKPKKLESAGSDKNCNIGQSSESHPCTMTEEWQTPYKETVEKLRELEDAYQRGKKAHDAMLGYANTAYAVNTKVEQE

SEQ ID NO: 9 - DNA, 1104 bp, synthetic construct
  source 1..1104: mol_type = other DNA
atgggcagcagccatcatcatcatcatcacagcagcggcctggtgccgcgcggcagccatatgttgttagtaattggcagtgaggataaccgtgttccaggagacaagaagttgacgaaagagggagcagcggcactctgtaaaatgaagcatcttgctgataaagttgcaaaggagagatcacaagagttaaaggatagaactcaaaattttgctggttatatagagttcgagttgtatagaatagattattggttggaaaagctgaacggtccgaaggggcgaaaagacggttatgctaagctgtctgactctgatatagaaaaagtaaaggagatattcaacaaggcaaaagatggaataactaagcaactccctgaggcaaagaaagccggtgaggaagctggaaaactacatactgaagtgaagaaggccgcggagaatgcacgtggacaggatcttgatgacgatacggcgaagtccactggcttgtacagggtgctgaattggtactgcattactaaagaagaaaggcacaacgctacccctaactgtgacggaatccaattcagaaagcattatctgtcggtgaatagaagcgctattgactgcagcagcaccagctatgaggagaattatgattggtctgcgaacgcgctgcaggtggccttaaatagctgggaggatgtgaagccaaaaaaattggagtcagccgggtcggacaagaattgcaacatcggccaatcttccgagagccatccatgcaccatgacagaggagtggcagactccatacaaggaaactgtcgaaaagctaagggaacttgaggatgcgtaccaaaggggcaagaaggctcatgatgctatgttgggttacgctaataccgcatatgctgtgaacacgaaagtggaacaggaaaagccgctgacagaggtgatagcggcagctaaggaagcagggaaaaagggcgcgaaaattataatacccgcagctgccccagcaacaccgactaacagcacaaaaaatgatgatagtgctccaaccgagcatgttgatagagggattgcaacaaatgaaacacaggtggaagttggtattgattaa

SEQ ID NO: 10 - protein, 367 aa, synthetic construct
MGSSHHHHHHSSGLVPRGSHMLLVIGSEDNRVPGDKKLTKEGAAALCKMKHLADKVAKERSQELKDRTQNFAGYIEFELYRIDYWLEKLNGPKGRKDGYAKLSDSDIEKVKEIFNKAKDGITKQLPEAKKAGEEAGKLHTEVKKAAENARGQDLDDDTAKSTGLYRVLNWYCITKEERHNATPNCDGIQFRKHYLSVNRSAIDCSSTSYEENYDWSANALQVALNSWEDVKPKKLESAGSDKNCNIGQSSESHPCTMTEEWQTPYKETVEKLRELEDAYQRGKKAHDAMLGYANTAYAVNTKVEQEKPLTEVIAAAKEAGKKGAKIIIPAAAPATPTNSTKNDDSAPTEHVDRGIATNETQVEVGID

SEQ ID NO: 11 - protein, 346 aa, synthetic construct
LLVIGSEDNRVPGDKKLTKEGAAALCKMKHLADKVAKERSQELKDRTQNFAGYIEFELYRIDYWLEKLNGPKGRKDGYAKLSDSDIKKVKEIFEKAKDGITKQLPEAKKAAEEAEKLHQEVKEAAEKARGQDLDDDTAKSTGLYRVLNWYCITKEERHNATPNCDGIQFRKHYLSVNRSAIDCSSTSYEENYDWSANALQVALNSWEDKKPKKLESAGSDKNCNIGQSSESHPCTMTEEWQTHYKETIEKLRELEEAYQRGKKAHDDMLGYANTAYAVNTKVEQEKPLSEVIAAAKEAGKKGAKIIIPAAAPATPTNSTKNDDSAPTEHVDRGIATNETQVEVGID

SEQ ID NO: 12 - protein, 237 aa, synthetic construct
YIEFELYRIDYWLEKLNGPKGRKDGYAKLSDSDIKKVKEIFEKAKDGITKQLPEAKKAAEEAEKLHQEVKEAAEKARGQDLDDDTAKSTGLYRVLNWYCITKEERHNATPNCDGIQFRKHYLSVNRSAIDCSSTSYEENYDWSANALQVALNSWEDKKPKKLESAGSDKNCNIGQSSESHPCTMTEEWQTHYKETIEKLRELEEAYQRGKKAHDDMLGYANTAYAVNTKVEQEKPLS

SEQ ID NO: 13 - protein, 365 aa, synthetic construct
MMKYLLVFAIIATRIPVLLVIGSEDNRVPGDKKLTKEGAAALCKMKHLADKVAKERSQELKDRTQNFAGYIEFELYRIDYWLEKLNGPKGRKDGYAKLSDSDIKKVKEIFEKAKDGITKQLPEAKKAAEEAEKLHQEVKEAAEKARGQDLDDDTAKSTGLYRVLNWYCITKEERHNATPNCDGIQFRKHYLSVNRSAIDCSSTSYEENYDWSANALQVALNSWEDKKPKKLESAGSDKNCNIGQSSESHPCTMTEEWQTHYKETIEKLRELEEAYQRGKKAHDDMLGYANTAYAVNTKVEQEKPLSEVIAAAKEAGKKGAKIIIPAAAPATPTNSTKNDDSAPTEHVDRGIATNETQVEVGIDAD

SEQ ID NO: 14 - protein, 286 aa, synthetic construct
SEDNRVPGDKKLTKEGAAALCKMKHLADKVAKERSQELKDRTQNFAGYIEFELYRIDYWLEKLNGPKGRKDGYAKLSDSDIKKVKEIFEKAKDGITKQLPEAKKAAEEAEKLHQEVKEAAEKARGQDLDDDTAKSTGLYRVLNWYCITKEERHNATPNCDGIQFRKHYLSVNRSAIDCSSTSYEENYDWSANALQVALNSWEDKKPKKLESAGSDKNCNIGQSSESHPCTMTEEWQTHYKETIEKLRELEEAYQRGKKAHDDMLGYANTAYAVNTKVEQEKPLSEV

SEQ ID NO: 15 - DNA, 1104 bp, synthetic construct
  source 1..1104: mol_type = other DNA
atgggcagcagccatcatcatcatcatcacagcagcggcctggtgccgcgcggcagccatatgctgctggtgattggcagcgaagataaccgcgtgccgggcgataaaaaactgaccaaagaaggcgcggcggcgctgtgcaaaatgaaacatctggcggataaagtggcgaaagaacgcagccaagaactgaaagatcgcacgcagaactttgcgggctatattgaatttgaactgtatcgcattgattattggctggaaaaactgaacggcccgaaaggccgcaaagatggctatgcgaaactgagcgatagcgatattaaaaaagtgaaagaaatttttgaaaaagcgaaagatggcattaccaaacagctgccggaagcgaaaaaagcggcggaagaagcggaaaaactgcatcaagaagtgaaagaagcggcggaaaaagcgcgcggccaagatctggatgatgataccgcgaaaagcaccggcctgtatcgcgtgctgaactggtattgcattaccaaagaagaacgccataacgcgaccccgaactgcgatggcattcagtttcgcaaacattatctgagcgtgaaccgcagcgcgattgattgcagcagcacgagctatgaagaaaactatgattggagcgcgaacgcgctgcaagtggcgctgaacagctgggaagataaaaaaccgaaaaaactggaaagcgcgggcagcgataaaaactgcaacattggtcagagcagcgaaagccatccgtgcaccatgaccgaagaatggcagacccattataaagaaaccattgaaaaactgcgcgaactggaagaagcgtatcagcgcggcaaaaaagcgcatgatgatatgctgggctatgcgaacaccgcgtatgcggtgaacaccaaagtggaacaagaaaaaccgctgagcgaagtgattgcggcggcgaaagaagcgggcaaaaaaggcgcgaaaattattattccggcggcggcgccggcgaccccgaccaacagcaccaaaaacgatgatagcgcgccgaccgaacatgtggatcgcggcattgcgaccaacgaaacccaagtggaagtgggcattgattaa

SEQ ID NO: 16 - protein, 367 aa, synthetic construct
MGSSHHHHHHSSGLVPRGSHMLLVIGSEDNRVPGDKKLTKEGAAALCKMKHLADKVAKERSQELKDRTQNFAGYIEFELYRIDYWLEKLNGPKGRKDGYAKLSDSDIKKVKEIFEKAKDGITKQLPEAKKAAEEAEKLHQEVKEAAEKARGQDLDDDTAKSTGLYRVLNWYCITKEERHNATPNCDGIQFRKHYLSVNRSAIDCSSTSYEENYDWSANALQVALNSWEDKKPKKLESAGSDKNCNIGQSSESHPCTMTEEWQTHYKETIEKLRELEEAYQRGKKAHDDMLGYANTAYAVNTKVEQEKPLSEVIAAAKEAGKKGAKIIIPAAAPATPTNSTKNDDSAPTEHVDRGIATNETQVEVGID

SEQ ID NO: 17 - protein, 21 aa, synthetic construct
MGSSHHHHHHSSGLVPRGSHM

SEQ ID NO: 18 - protein, 7 aa, synthetic construct
TAKSTGL

(19) United States
(12) Patent Application Publication
(10) Pub. No.: US 2026/0055148 A1
(43) Pub. Date: Feb. 26, 2026

(54) PROTEINS FOR USE IN THE TREATMENT OF COMPLEMENT DYSREGULATION DISORDERS

(71) Applicant: USTAV ORGANICKE CHEMIE A BIOCHEMIE AV CR, V. V. I., Praha 6 (CZ)

(72) Inventors: Sebastian ZOLL, Praha (CZ); Hagen SÜLZEN, Praha (CZ)

(21) Appl. No.: 19/105,378

(22) PCT Filed: Aug. 23, 2023
(86) PCT No.: PCT/CZ2023/050053

§ 371 Date: Feb. 21, 2025

(30) Foreign Application Priority Data
Aug. 24, 2022 (EP) 22192040.8
Feb. 10, 2023 (EP) 23156104.4
Feb. 10, 2023 (EP) 23156132.5

(51) Int. Cl. C07K 14/44; A61K 38/00

(52) U.S. Cl. CPC C07K 14/44; A61K 38/00

(57) ABSTRACT

The present invention relates to a protein derived from the extracellular domain of the surface protein ISG65 (invariant surface glycoprotein 65 (Tbg.972.2.1600) of the human parasite Trypanosoma brucei gambiense. The ISG65-derived protein is useful in the treatment of complement dysregulation diseases. In one aspect, the present invention provides a novel thermostable mutant of ISG65.

### FIELD OF ART

The present invention relates to proteins for use in the treatment of complement dysregulation disorders.

### BACKGROUND ART

The complement cascade is a part of the innate immune system which complements the ability of antibodies and phagocytes to clear microbes and damaged cells and promote inflammation. The complement cascade is activated by three biochemical pathways: the classical pathway, the alternative pathway and the lectin pathway. The alternative pathway is responsible for the majority of terminal pathway activation. The alternative pathway is continuously activated at a low level, due to spontaneous hydrolysis of mildly unstable C3, generating C3b. C3b attaches to cell surfaces where it forms the nidus for formation of the C3 and C5 concertases which eventually lead to assembly of the membrane attack complex in the terminal pathway.

Excessive activation of complement proteins is often discovered to be the reason for many diseases. These include e.g. autoimmune diseases, Alzheimer's syndrome, schizophrenia, atypical hemolytic-uremic syndrome, angioedema, macular degeneration, and Crohn's disease (Tichaczek-Goska D.: Adv Clin Exp Med 2012 January-Feb; 21 (1): 105-14). Atypical hemolytic uremic syndrome (aHUS), C3 glomerulopathy (C3G), and paroxysmal nocturnal hemoglobinuria (PNH) are prototypical disorders of complement dysregulation (Wong E. K. S, and Kavangh D.: Semin Immunopathol 2018 January: 40 (1): 49-64, doi: 10.1007/s00281-017-0663-8).

Mutations in the thioester domain (TED) of human C3b or in the C3b binding domain of human factor H, a negative regulator of the complement cascade, result in a loss of cellular self-recognition, uncontrolled lysis of red blood cells and consequently kidney failure. Treatment of these diseases currently requires Eculizumab, a monoclonal antibody which targets C5. Due to the prohibitive cost of this drug cheaper alternatives are much needed.

### SUMMARY OF THE INVENTION

The present invention is based on the finding that the extracellular domain of the surface protein ISG65 (invariant surface glycoprotein 65 (Tbg.972.2.1600)) of the human parasite Trypanosoma brucei gambiense is a complement receptor which targets the complement cascade at several critical intervention points. The extracellular domain of ISG65 binds to native C3, to its reactive activation products C3b and C3 (H₂O), and to C3d, but not to C3c. Addition of the extracellular domain of ISG65 to human serum resulted in a concentration-dependent decrease in haemolysis of erythrocytes indicating an inhibition of the complement cascade. This inhibition could only be observed for the alternative pathway, but not the classical pathway (CP). An inhibition of the lectin pathway (LP) was not tested directly, but is considered implausible since CP and LP share the same C3b-containing component. This leads to the conclusion that ISG65-mediated inhibition is selective for the alternative pathway. We further show that inhibition takes place at the level of the AP C5 convertase. C3b serves as a central hub in the complement cascade and therefore its inhibition displays a more comprehensive approach for treatment of complement dysregulations than is targeting several downstream effector pathways (e.g. C5 or C9 polymerization). Also, unlike monoclonal antibodies, the ISG65 extracellular domain targets several interaction partners within the cascade, which potentially increases its potency.

The present invention thus provides an ISG65-derived protein for use as a medicament, in particular for use in the treatment of complement dysregulation diseases. “Dyregulation” herein refers in particular to excessive activation.

More specifically, the invention provides the ISG65-derived protein for use in the treatment of a disease selected from atypical hemolytic uremic syndrome (aHUS), C3 glomerulopathy (C3G), paroxysmal nocturnal hemoglobinuria (PNH), autoimmune diseases, Alzheimer's syndrome, schizophrenia, angioedema, macular degeneration, and Crohn's disease.

Native extracellular domain of ISG65 has the amino acid sequence SEQ ID NO: 1:

MMKYLLVFAIIATRIPVLLVIGSEDNRVPGDKKLTKEGAAALCKMKHLAD
KVAKERSQELKDRTQNFAGYIEFELYRIDYWLEKLNGPKGRKDGYAKLSD
SDIEKVKEIFNKAKDGITKQLPEAKKAGEEAGKLHTEVKKAAENARGQDL
DDDTAKSTGLYRVLNWYCITKEERHNATPNCDGIQFRKHYLSVNRSAIDC
SSTSYEENYDWSANALQVALNSWEDVKPKKLESAGSDKNCNIGQSSESHP
CTMTEEWQTPYKETVEKLRELEDAYQRGKKAHDAMLGYANTAYAVNTKVE
QEKPLTEVIAAAKEAGKKGAKIIIPAAAPATPTNSTKNDDSAPTEHVDRG
IATNETQVEVGIDADFDSLLDATEAAEVTRRHQRTAM.

The extracellular domain of ISG65 consists mainly of an approx. 80 Å (8 nm) long 3-helix bundle. Extensive loop structures stabilised by disulphide bonds establish the head domain which is located near the N-terminus of the protein and membrane-distal in the mature, membrane-embedded protein. The membrane-proximal parts of bundle helices 1 and 3 contain the majority of C3-TED interacting residues. Another interaction site is located in a loop connecting helices 2 and 3, just C-terminal of a short 3₁₀-helix.

The three interaction sites are underlined in SEQ ID NO. 1, and are the following:

(SEQ ID NO: 2)
site 1: 70-YIEFELYRIDYWLEKLNGPKGRKDGYAK-97
(SEQ ID NO: 3)
site 2: 210-DWSA-213
(SEQ ID NO: 4)
site 3: 290-NTAYAVNTKVEQE-302

Within the interaction sites, the identified crucial interface residues are: Tyr70, Phe73, Arg77, Trp81, Lys97; Trp211; Asn290, Tyr293, Thr297, Glu302.

The sequence of the extracellular domain of ISG65 used in the experiments presented in this patent application was SEQ ID NO: 5:

(SEQ ID NO: 5)
LLVIGSEDNRVPGDKKLTKEGAAALCKMKHLADKVAKERSQELKDRTQNF
AGYIEFELYRIDYWLEKLNGPKGRKDGYAKLSDSDIEKVKEIENKAKDGI
TKQLPEAKKAGEEAGKLHTEVKKAAENARGQDLDDDTAKSTGLYRVLNWY
CITKEERHNATPNCDGIQFRKHYLSVNRSAIDCSSTSYEENYDWSANALQ
VALNSWEDVKPKKLESAGSDKNCNIGQSSESHPCTMTEEWQTPYKETVEK
LRELEDAYQRGKKAHDAMLGYANTAYAVNTKVEQEKPLTEVIAAAKEAGK
KGAKIIIPAAAPATPTNSTKNDDSAPTEHVDRGIATNETQVEVGID,
further containing an affinity tag:
(SEQ ID NO: 11)
MGSSHHHHHHSSGLVPRGSHM.

The longest, truncated sequence of the extracellular domain of ISG65 that can be produced recombinantly is SEQ ID NO: 6:

(SEQ ID NO: 6)
MMKYLLVFAIIATRIPVLLVIGSEDNRVPGDKKLTKEGAAALCKMKHLADK
VAKERSQELKDRTQNFAGYIEFELYRIDYWLEKLNGPKGRKDGYAKLSDSD
IEKVKEIENKAKDGITKQLPEAKKAGEEAGKLHTEVKKAAENARGQDLDDD
TAKSTGLYRVLNWYCITKEERHNATPNCDGIQFRKHYLSVNRSAIDCSSTS
YEENYDWSANALQVALNSWEDVKPKKLESAGSDKNCNIGQSSESHPCTMTE
EWQTPYKETVEKLRELEDAYQRGKKAHDAMLGYANTAYAVNTKVEQEKPLT
EVIAAAKEAGKKGAKIIIPAAAPATPTNSTKNDDSAPTEHVDRGIATNETQ
VEVGIDAD.

In some embodiments, a sequence of the extracellular domain of ISG65 that can be produced recombinantly is SEQ ID NO: 7:

(SEQ ID NO: 7)
SEDNRVPGDKKLTKEGAAALCKMKHLADKVAKERSQELKDRTQNFAGYIE
FELYRIDYWLEKLNGPKGRKDGYAKLSDSDIEKVKEIFNKAKDGITKQLP
LPEAKKAGEEAGKLHTEVKKAAENARGQDLDDDTAKSTGLYRVLNWYCIT
KEERHNATPNCDGIQFRKHYLSVNRSAIDCSSTSYEENYDWSANALQVAL
NSWEDVKPKKLESAGSDKNCNIGQSSESHPCTMTEEWQTPYKETVEKLRE
LEDAYQRGKKAHDAMLGYANTAYAVNTKVEQEKPLTEV.

Engineered variants of the native ISG65 extracellular domain sequence can be used, e.g. variants having a higher thermostability, lyophilizable variants, etc., or suitable isoforms (several native isoforms of ISG65 are known to date). These variants and isoforms are herein included in the term ISG65-derived protein “.

The term ISG65-derived protein” herein refers to proteins having the length of up to 500 amino acid residues and containing an amino acid sequence having at least 90% identity, preferably at least 95% identity, to the sequence SEQ ID NO: 8, wherein the underlined regions are conserved:

(SEQ ID NO: 8)
GYIEFELYRIDYWLEKLNGPKGRKDGYAKLSDSDIEKVKEIENKAKDGIT
KQLPEAKKAGEEAGKLHTEVKKAAENARGQDLDDDTAKSTGLYRVLNWYC
ITKEERHNATPNCDGIQFRKHYLSVNRSAIDCSSTSYEENYDWSANALQV
ALNSWEDVKPKKLESAGSDKNCNIGQSSESHPCTMTEEWQTPYKETVEKL
RELEDAYQRGKKAHDAMLGYANTAYAVNTKVEQE.

More preferably, the ISG65-derived protein is a protein having the length of up to 500 amino acid residues and containing an amino acid sequence having at least 90% identity, preferably at least 95% identity, to the sequence SEQ ID NO: 7, wherein the underlined regions are conserved.

In some embodiments, the ISG65-derived protein is a protein having the length of up to 500 amino acid residues and containing an amino acid sequence having at least 90% identity, preferably at least 95% identity, to the sequence SEQ ID NO: 5, wherein the underlined regions are conserved.

In some embodiments, the ISG65-derived protein is a protein having the length of up to 500 amino acid residues and containing an amino acid sequence having at least 90% identity, preferably at least 95% identity, to the sequence SEQ ID NO: 6, wherein the underlined regions are conserved.

In a particular aspect, the present invention provides a new ISG65-derived protein exhibiting increased thermostability. The new protein carries several mutations (FIG. 10) and shows a significant and unexpected increase in the melting temperature (ISG65 M2 by 14.7° C.), compared to the native ISG65 sequence (FIG. 11). For practical use, proteins with a high stability are particularly beneficial, allowing to produce and store medical products.

The sequence of the extracellular domain of the thermostable mutant of ISG65 used in the experiments presented in this application was SEQ ID NO: 16, which consists of SEQ ID NO: 11, with affinity tag SEQ ID NO: 17. This sequence is referred to herein as ISG65 M2.

(SEQ ID NO: 11)
LLVIGSEDNRVPGDKKLTKEGAAALCKMKHLADKVAKERSQELKDRTQNF
AGYIEFELYRIDYWLEKLNGPKGRKDGYAKLSDSDIKKVKEIFEKAKDGI
TKQLPEAKKAAEEAEKLHQEVKEAAEKARGQDLDDDTAKSTGLYRVLNWY
CITKEERHNATPNCDGIQFRKHYLSVNRSAIDCSSTSYEENYDWSANALQ
VALNSWEDKKPKKLESAGSDKNCNIGQSSESHPCTMTEEWQTHYKETIEK
LRELEEAYQRGKKAHDDMLGYANTAYAVNTKVEQEKPLSEVIAAAKEAGK
KGAKIIIPAAAPATPTNSTKNDDSAPTEHVDRGIATNETQVEVGID
(SEQ ID NO: 17)
MGSSHHHHHHSSGLVPRGSHM

Residues mutated in the thermostable ISG65 M2 mutant are: Glu104Lys, Asn111Glu, Gly128Ala, Gly 132Glu, Thr136Gln, Lys140Glu, Asn144Lys, Val226Lys, Pro260His, Val265Ile, Asp273Glu, Ala284Asp, Thr306Ser

These residues are bolded-underlined in the sequences SEQ ID NO: 11-14.

Overall, 13 amino acids were mutated in the new thermostable mutant ISG65 M2 in comparison to wild type ISG65. Their positions are marked in a sequence alignment between ISG65 M2 and wild type ISG65 (FIG. 10) and were mapped onto a structural model of ISG65 M2 (FIGS. 12A and 12B).

The term thermostable ISG65 mutant” or thermostable mutant of ISG65″ herein refers to proteins having the length of up to 500 amino acid residues and containing an amino acid sequence having at least 98% identity, preferably at least 99% identity, to the sequence SEQ ID NO: 12, wherein the underlined and bolded-underlined regions are conserved:

(SEQ ID NO: 12)
YIEFELYRIDYWLEKLNGPKGRKDGYAKLSDSDIKKVKEIFEKAKDGITK
QLPEAKKAAEEAEKLHQEVKEAAEKARGQDLDDDTAKSTGLYRVLNWYCI
TKEERHNATPNCDGIQFRKHYLSVNRSAIDCSSTSYEENYDWSANALQVA
LNSWEDKKPKKLESAGSDKNCNIGQSSESHPCTMTEEWQTHYKETIEKLR
ELEEAYQRGKKAHDDMLGYANTAYAVNTKVEQEKPLS.

In some embodiments, preferred embodiments of thermostable ISG65 mutant” or thermostable mutant of ISG65″ are sequences containing or consisting of sequences SEQ ID NO: 11, 13 and 14.

The longest, truncated sequence of the thermostable ISG65 mutant that can be produced recombinantly is SEQ ID NO: 13:

MMKYLLVFAIIATRIPVLLVIGSEDNRVPGDKKLTKEGAAALCKMKHLAD
KVAKERSQELKDRTQNFAGYIEFELYRIDYWLEKLNGPKGRKDGYAKLSD
SDIKKVKEIFEKAKDGITKQLPEAKKAAEEAEKLHQEVKEAAEKARGQDL
DDDTAKSTGLYRVLNWYCITKEERHNATPNCDGIQFRKHYLSVNRSAIDC
SSTSYEENYDWSANALQVALNSWEDKKPKKLESAGSDKNCNIGQSSESHP
CTMTEEWQTHYKETIEKLRELEEAYQRGKKAHDDMLGYANTAYAVNTKVE
QEKPLSEVIAAAKEAGKKGAKIIIPAAAPATPTNSTKNDDSAPTEHVDRG
IATNETQVEVGIDAD.

In some embodiments, the invention includes proteins having the length of up to 500 amino acid residues and containing an amino acid sequence having at least 98% identity, preferably at least 99% identity, to the sequence SEQ ID NO: 13, wherein the underlined and bolded-underlined regions are conserved.

In some embodiments, a sequence of the extracellular domain of the thermostable ISG65 mutant that can be produced recombinantly is SEQ ID NO: 14:

SEDNRVPGDKKLTKEGAAALCKMKHLADKVAKERSQELKDRTQNFAGYIE
FELYRIDYWLEKLNGPKGRKDGYAKLSDSDIKKVKEIFEKAKDGITKQLP
EAKKAAEEAEKLHQEVKEAAEKARGQDLDDDTAKSTGLYRVLNWYCITKE
ERHNATPNCDGIQFRKHYLSVNRSAIDCSSTSYEENYDWSANALQVALNS
WEDKKPKKLESAGSDKNCNIGQSSESHPCTMTEEWQTHYKETIEKLRELE
EAYQRGKKAHDDMLGYANTAYAVNTKVEQEKPLSEV.

In some embodiments, the invention includes proteins having the length of up to 500 amino acid residues and containing an amino acid sequence having at least 98% identity, preferably at least 99% identity, to the sequence SEQ ID NO: 14, wherein the underlined and bolded-underlined regions are conserved.

The length of the ISG65-derived protein is up to 500 amino acid residues, more preferably up to 450 amino acid residues, in some embodiments up to 400 amino acid residues. The protein may be extended on one or both termini of the herein shown sequence. The extension sequence may contain, for example, an affinity tag, or a sequence with at least 90% identity, to the corresponding ISG65 region.

Affinity tags are known in the art, and include, for example: ALFA-tag, AviTag, C-tag, polyglutamare tag, polyarginine tag, E-tag, FLAG-tag, HA-tag, His-tag, Myc-tag, NE-tag, Rho1D4-tag, S-tag, Softag 1, Softag3, Spot-tag, Strep-tag, T7-tag, TC tag, Ty tag, V5 tag, VSV-tag, Xpress tag, Isopeptag, Spy Tag, Snoop Tag, DogTag, or Sdy Tag.

The term identity” refers to the number of identical residues over the total length of the reference sequence. The reference alignment is the CLUSTAL O (1.2.4) multiple sequence alignment (https://www.ebi.ac.uk/Tools/msa/clustalo/).

The percentage of identity lower than 100% refers to sequences differing from the reference sequence by terminal extensions, terminal deletions, point mutations, insertions and deletions within the sequence.

The present invention further provides a pharmaceutical formulation containing the ISG65-derived protein and at least one pharmaceutically acceptable excipient. The excipients may include solvents, such as water or a buffer, stabilizers, surfactants, binders, fillers and glidants. The formulations may be liquid or solid formulations. In solid formulations, the protein may preferably be lyophilized.

### BRIEF DESCRIPTION OF DRAWINGS

FIG. 1: Chromatogram showing purification of the ISG65: C3b complex (˜220 kDa) using size exclusion chromatography. Complex formation of ISG65 with C3b was confirmed by analysing fractions underneath the main peak (marked with horizontal bar) on an SDS gel.

FIGS. 2A to 2E: Surface plasmon resonance sensorgrams showing the kinetics of ISG65 binding to C3d (FIG. 2A), C3 (H₂O) (FIG. 2B), C3b (FIG. 2C), C3 (FIG. 2D), and C3c (FIG. 2E). Biotinylated ISG65 (ligand) was immobilised, and different concentrations of indicated analytes were allowed to flow over ISG65.

FIGS. 3A and 3B: Haemolysis assay (AP50) measuring the lysis of red blood cells in human serum in presence of ISG65 (top) and ISG75 (bottom) following activation of the alternative complement pathway (n=3: ±std S. D: ** indicates p≤0.01: ***indicates p≤0.001). ISG65 (FIG. 3A) shows a significant, concentration-dependent inhibition of erythrocyte lysis. Addition of ISG75 (FIG. 3B) to serum has no significant effect on erythrocyte lysis. Protein concentration increases in direction of the arrow. The highest concentration used for both proteins is 7.5 M.

FIG. 4: Haemolysis assay (CH50) measuring the lysis of red blood cells in human serum in presence of ISG65, ISG75 and without added inhibitors (NHS, normal human serum: solid black) following activation of the classical complement pathway (n=4: +std S. D). At 24 μM, both ISG65 and ISG75 show no significant inhibition of erythrocyte lysis. Low complement activity serum (n=3, ***indicates p≤0.001) is shown as reference.

FIGS. 5A to 5C: Organisation of C3 complexes and their possible interactions with ISG65. Top (FIG. 5A), ISG65-bound C3b can interact with factor B (fB) followed by factor D (fD), allowing the formation of the C3 proconvertase C3bfB. Middle (FIG. 5B), ISG65-bound AP C3 convertase would be able to bind the C3 substrate according to the model proposed by Rooijakkers et al. (Rooijakkers SH, Ruyken M, Roos A, et al. Immune evasion by a staphylococcal complement inhibitor that acts on C3 convertases. Nat Immunol. 2005: 6 (9): 920-927, doi: 10.1038/ni1235). Bottom (FIG. 5C), ISG65-bound C3 substrate would be able to interact with C3bBb. ISG65-interacting proteins are underlined in the titles.

FIG. 6: ISG65 does not prevent formation of the AP C3 proconvertase. SPR sensorgrams showing AP proconvertase formation in presence of ISG65. C3b was immobilised onto the chip and ISG65 and factor B subsequently injected. Control injections were performed with ISG65 or factor B only. Beginning and end of the injections are marked with arrows (ISG65: empty, factor B: full).

FIG. 7: Adapted AP50 test using C5 and C3 depleted human sera. Binding of ISG65 to assembled AP C5 convertase C3bBbC3b significantly reduces haemolysis. No reduction in haemolysis could be detected for ISG75. Both proteins were used at 7.5 μM. Human sera depleted for either C5 (−C5) or C3 (−C3) do not cause haemolysis. Incubation of erythrocytes with C3-depleted serum followed by incubation with C5 depleted serum, reconstituted haemolytic activity. (n=3: + std S. D: * indicates p≤ 0.05**: indicates p≤0.01: ***indicates p≤0.001).

FIGS. 8A and 8B: Cryo-EM structures of ISG65 in complex with C3 and C3b. A (FIG. 8A). Cryo-EM density maps showing side views of ISG65: C3 (left), and ISG65: C3b (right) complexes at 2 different angles. C3 (b) domains are labelled. B (FIG. 8B). Cartoon representation of ISG65: C3 and ISG65: C3b. Interacting domains are indicated in black.

FIGS. 9A and 9B: ISG65 binding to C3 ANA/C3a. A (FIG. 9A). Close-up view of the interface between ISG65 and C3-ANA showing residues that engage in hydrogen bonds-ISG65 K97 and ANA E689 as well as ISG65 E302 and ANA G717. B (FIG. 9B). SPR sensorgrams of interaction between ISG65 (ligand) and C3a (analyte).

FIG. 10. Multiple sequence alignment of ISG65 wt and the mutated protein ISG65 M2. In total. 13 residues are changed in ISG65 M2 compared to ISG65 wt. Residues are compared based on their physio-chemical proletaries and declared as identical (*). highly similar (:), similar (.) and dissimilar ( ) The alignment was generated using the software Clustal O (mega) (https://www.ebi.ac.uk/Tools/msa/clustalo/).

FIG. 11. Melting curves of ISG65 wt and ISG65 M2. The melting temperature (Tm) of ISG65 M2 is increased by 14.7° C., compared to ISG65 wt. Curves and melting temperatures were calculated from circular dichroism spectra at 222 nm wavelength. The melting temperatures were determined from the inflection points of the sigmoidal unfolding curves. 0, molar ellipticity (deg cm² dmol⁻¹) FIGS. 12A and 12B. Structural comparison of ISG65 M2 (FIG. 12A) and ISG65 wt (FIG. 12B) in 2 different orientations. Positions of the thermostability improving mutations in ISG65 M2 are indicated. All mutations are located outside the C3 binding site of ISG65. The first letter represents the mutated residue in ISG65 M2, the number represents the position in the protein and the last letter the residue that was mutated in the wt protein. To obtain a better visual overview about the positions of the introduced mutations, the model of ISG65 M2 was computationally predicted using the software AlphaFold2 (top) (Jumper J, Evans R, Pritzel A, et al. Highly accurate protein structure prediction with AlphaFold. Nature. 2021; 596 (7873): 583-589, doi: 10.1038/s41586-021-03819-2.) The model of ISG65 wt (bottom) is derived from experimental data (cryo-EM). ISG65 wt and ISG65 M2 are highly similar and can be aligned with an RSMD=2.1 Å (Pymol Align. Schrodinger. L., & DeLano. W. (2020). PyMOL. Retrieved from http://www.pymol.org/pymol). This implies that the indicated mutations are unlikely to change the overall fold and the functional properties of ISG65. RMSD, root-mean-square deviation of atomic positions.

FIG. 13. Chromatogram showing purification of the C3b: ISG65 M2 complex (˜220 kDa) using size exclusion chromatography on Superdex S200 10/300. Complex formation of ISG65 M2 with C3b was confirmed by analysing fractions underneath the main peak (marked with horizontal bar) on a reducing SDS gel. * C3b alpha-chain. ** C3b beta-chain. ***ISG65.

FIGS. 14A to 14D. FACS of mouse splenocytes with ISG65 and its binding partner demonstrates the absence of neutralizing antibodies. Live, single B-cells have been analysed for AF 594 presence via flow cytometry using the Y610 channel. Fraction of cells for each gate are indicated in percent. A (FIG. 14A). Negative control. B-cells positive for AF594 in the first round of labelling retain no ISG65 AF594 after washing. B (FIG. 14B). Re-incubating the pre-selected B-cells with ISG65AF594 results in 24.5% AF594 positive, single B-cells. C (FIG. 14C). Incubation with the ISG65AF594: C3d complex results in 19.4% of AF 594 positive, single B-cells. D (FIG. 14D), Incubation with the ISG65AF594: Fab complex reduces the fraction of AF594 positive, single B-cells to 4.76%. Abbreviations: -A (pulse area), -H (pulse height). SSC (side-scattering). FSC (forward scattering).

FIG. 15. ISG65 stability in Human plasma. Stability of ISG65 in human plasma was assessed over a time course of 11 days. Abundance and quantity of ISG65 was determined via Western Blot with polyclonal mouse anti-ISG65 serum and a fluorescent anti-mouse IgG secondary antibody. The imaged Western Blot is displayed, the molecular weight marker is annotated on the left and on the right side (in kDa). Time points of sample acquisition are indicated above the respective lane. The fluorescence intensity, in comparison to the reference sample (0 hours), is plotted for each sample, underneath the corresponding protein band.

FIGS. 16A and 16B. Functional assessment of ISG65 and ISG65 M2 stability in human plasma via its capacity to inhibit the alternative complement system. A (FIG. 16A). The relative AP50 (plasma fraction resulting in 50% of maximal hemolysis) has been plotted for day 0 and day 3 of human plasma, incubated with and without ISG65, at 37° C. For each day, both reference and sample plasma are shown. At both observed timepoints. ISG65 did noticeably inhibit the complement activity in comparison to the reference sample. (n=2 [technical replicates], error bars indicate standard error of the mean (SEM)). B (FIG. 16B). The relative AP50 (plasma fraction resulting in 50% of maximal hemolysis) has been plotted for day 0 and day 3 of human plasma, incubated with and without ISG65 M2, at 37° C. For each day, both reference and sample plasma are shown. Within the error the inhibitory effect of ISG65 did not change of the course of 3 days (n=2[technical replicates], error bars indicate standard error of the mean (SEM)).

### DETAILED DESCRIPTION OF THE INVENTION

In this chapter, the term “ISG65” refers to the tested extracellular domain of ISG65 (His-ISG₆₅₁₈₋₃₆₃).

The term “ISG65 M2” refers to the particularly thermostable, preferred, ISG65-derived protein.

### Cloning, Expression and Purification of ISG65

ISG65 A DNA fragment (Genewiz) encoding amino acids (aa) 18-363 of ISG65 from T. b. gambiense (Tbg.972.2.1600) was codon optimised for bacterial expression and cloned into the pET15b plasmid using Gibson assembly method (New England Biolabs). For recombinant expression in E, coli T7 shuffle cells (New England Biolabs), a plasmid encoding an N-terminal hexa-histidine-tag (His-ISG65₁₈₋₃₆₃) and another with an additional C-terminal Avi-tag (His-ISG₆₅₁₈₋₃₆₃-Avi) were generated. Amino acids substitutions were introduced into the His-ISG₆₅₁₈₋₃₆₃ construct using the Q5 site-directed mutagenesis kit (New England Biolabs). For all ISG65 variants, the same expression and purification protocol was employed. Proteins were produced overnight at 22° C., after induction of protein expression with 1 mM Isopropyl-b-D-thiogalacto-pyranoside (IPTG). Soluble protein was purified from bacterial lysate by immobilised metal-affinity chromatography (IMAC) using nickel-nitrilotriacetic acid (NTA) beads (Qiagen) and gravity-flow columns (Bio-Rad). After IMAC, the purified proteins were concentrated using Amicon Ultra centrifugal filters (Merck Millipore) and subjected to size-exclusion chromatography using a Superdex 200 Increase 10/300 GL (GE Healthcare) equilibrated with 20 mM HEPES (pH 7.5) and 150 mM NaCl. The DNA sequence of the expression construct was SEQ ID NO: 9:

ATGGGCAGCAGCCATCATCATCATCATCACAGCAGCGGCCTGGTGCCGC
GCGGCAGCCATATGTTGTTAGTAATTGGCAGTGAGGATAACCGTGTTCC
AGGAGACAAGAAGTTGACGAAAGAGGGAGCAGCGGCACTCTGTAAAATG
AAGCATCTTGCTGATAAAGTTGCAAAGGAGAGATCACAAGAGTTAAAGG
ATAGAACTCAAAATTTTGCTGGTTATATAGAGTTCGAGTTGTATAGAAT
AGATTATTGGTTGGAAAAGCTGAACGGTCCGAAGGGGCGAAAAGACGGT
TATGCTAAGCTGTCTGACTCTGATATAGAAAAAGTAAAGGAGATATTCA
ACAAGGCAAAAGATGGAATAACTAAGCAACTCCCTGAGGCAAAGAAAGC
CGGTGAGGAAGCTGGAAAACTACATACTGAAGTGAAGAAGGCCGCGGAG
AATGCACGTGGACAGGATCTTGATGACGATACGGCGAAGTCCACTGGCT
TGTACAGGGTGCTGAATTGGTACTGCATTACTAAAGAAGAAAGGCACAA
CGCTACCCCTAACTGTGACGGAATCCAATTCAGAAAGCATTATCTGTCG
GTGAATAGAAGCGCTATTGACTGCAGCAGCACCAGCTATGAGGAGAATT
ATGATTGGTCTGCGAACGCGCTGCAGGTGGCCTTAAATAGCTGGGAGGA
TGTGAAGCCAAAAAAATTGGAGTCAGCCGGGTCGGACAAGAATTGCAAC
ATCGGCCAATCTTCCGAGAGCCATCCATGCACCATGACAGAGGAGTGGC
AGACTCCATACAAGGAAACTGTCGAAAAGCTAAGGGAACTTGAGGATGC
GTACCAAAGGGGCAAGAAGGCTCATGATGCTATGTTGGGTTACGCTAAT
ACCGCATATGCTGTGAACACGAAAGTGGAACAGGAAAAGCCGCTGACAG
AGGTGATAGCGGCAGCTAAGGAAGCAGGGAAAAAGGGCGCGAAAATTAT
AATACCCGCAGCTGCCCCAGCAACACCGACTAACAGCACAAAAAATGAT
GATAGTGCTCCAACCGAGCATGTTGATAGAGGGATTGCAACAAATGAAA
CACAGGTGGAAGTTGGTATTGATTAA.

The translated sequence, including the affinity tag, was SEQ ID NO: 10:

MGSSHHHHHHSSGLVPRGSHMLLVIGSEDNRVPGDKKLTKEGAAALCKM
KHLADKVAKERSQELKDRTQNFAGYIEFELYRIDYWLEKLNGPKGRKDG
YAKLSDSDIEKVKEIFNKAKDGITKQLPEAKKAGEEAGKLHTEVKKAAE
NARGQDLDDDTAKSTGLYRVLNWYCITKEERHNATPNCDGIQFRKHYLS
VNRSAIDCSSTSYEENYDWSANALQVALNSWEDVKPKKLESAGSDKNCN
IGQSSESHPCTMTEEWQTPYKETVEKLRELEDAYQRGKKAHDAMLGYAN
TAYAVNTKVEQEKPLTEVIAAAKEAGKKGAKIIIPAAAPATPTNSTKND
DSAPTEHVDRGIATNETQVEVGID.

Isg75 (Comparative) ISG75 was expressed and purified as previously described (doi: 10.3390/pathogens10081050 (2021)).
Human Complement Factor 3 Native C3 was purified from normal human serum (Sigma-Aldrich) (as described in doi: 10.1002/0471142735.im 1303s14 (2001)). Purification and generation of the fragments C3b and C3 (H₂O) were carried out as described (doi: 10.1016/0022-1759 (89) 90340-898 2 (1989)). In short, the thioester in C3 was hydrolysed using methylamine and deactivated by incubation with iodoacetamide. C3c and C3a were purchased from Complement Technology, Inc. C3d (His-C3d) was produced recombinantly in E, coli as described (doi: 10.1021/bi0101749 (2001)). Amino acid substitutions were introduced using the same method as for ISG65. For SPR assays, a C3d construct (His-C3d-Avi) with an N-terminal His-tag and a C-terminal Avi-tag was generated. Cloning of this construct into pET15b was done by the Gibson assembly method. Expression and purification of all C3d constructs were carried out in the same way as described for ISG65.

### Binding of ISG65 to Human C3

Initially, the interaction between the extracellular domain of ISG65 (His-ISG₆₅₁₈₋₃₆₃) and human C3 was identified using IMAC-assisted pulldown assay with the extracellular domain of ISG65 (His-ISG₆₅₁₈₋₃₆₃) and commercially obtained, non-heat inactivated human serum. 400 μL of settled Ni-NTA resin (Qiagen) were incubated with 200 μg of His-ISG₆₅₁₈₋₃₆₃, washed with 2.5 ml IMAC wash buffer (20 mM Tris, 500 mM NaCl, 20 mM Imidazole, pH8.0) and incubated with 2.5 ml of human serum. Subsequently the beads were washed again using the same buffer, before bound protein was eluted with in a total volume of 2 ml IMAC elution buffer (20 mM Tris, 500 mM NaCl, 400 mM Imidazole, pH8.0). 4 fractions were collected and analysed by SDS-PAGE and Coomassie-staining. All steps were carried out in gravity-flow columns (Biorad). ISG65 (without serum) and serum (without ISG65 as bait protein) were used as negative controls. Specific enrichment of C3 in the presence of ISG65 was confirmed by mass spectrometry (LC-MS/MS) form cut-out gel bands.

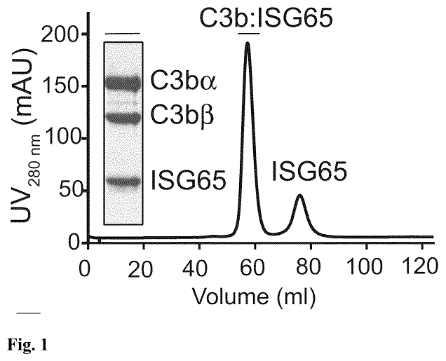

To further characterise this interaction and its stability, purified ISG65 was incubated in 3-fold molar excess with C3b and subjected to size exclusion chromatography. Two distinct peaks corresponding to the ISG65: C3b complex and excess ISG65 were observed. Analysis of peak fractions by SDS-PAGE revealed three bands that corresponded to ISG65 (39 kDa) as well as to the α- and β-chains of C3b (70, and, respectively 100 kDa), demonstrating that a stable complex was formed (FIG. 1). To further characterise the interaction, the complex was crosslinked using disuccinimidyl adipate (DSA). For cross-linking, proteins were transferred into buffer containing 20 mM HEPES (pH 7.5) and 150 mM NaCl. ISG65 and C3b were mixed in a 2:1 molar ratio, and freshly prepared DSA was added to the proteins at a hundred molar access. The reaction mixture was incubated at room temperature for 30 min and quenched with 10 molar access of ethanolamine. For EM-grid preparation, the cross-linked complex was also gel-filtered on Superdex 200 Increase 10/300 GL (GE Healthcare). SDS-PAGE analysis of the crosslinked sample showed two bands migrating at approximately 220 kDa and 39 kDa, consistent with crosslinked ISG65: C3b complex with a stochiometric ratio of 1:1 and free ISG65.

### Biophysical Characterisation of ISG65 Binding to C3 Fragments

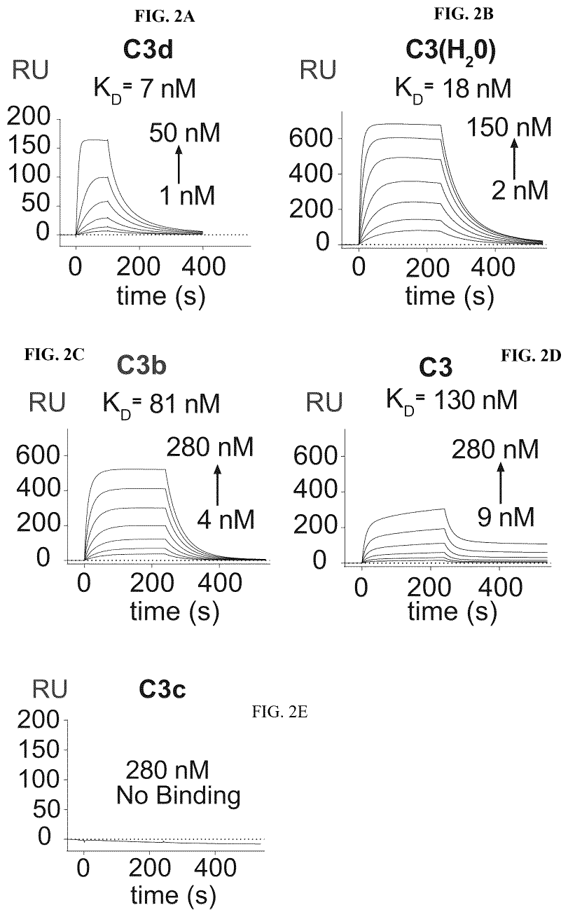

During the activation of the AP. C3 undergoes multiple proteolytic cleavages that result in an array of activated fragments, each with a specific role within the AP. To understand whether ISG65 can distinguish between these naturally occurring fragments, affinity measurements using surface plasmon resonance (SPR) were carried out (FIGS. 2A to 2E. Table 1). ISG65 extracellular domain was immobilised on the SPR sensor chip via its biotinylated C-terminus (His-ISG₆₅₁₈₋₃₆₃-Avi) while the different C3 fragments were applied to the chip as analytes. This coating strategy represented a good mimetic of the presentation of native ISG65 on the cell surface via its transmembrane anchor. Binding of the two major C3 fragments. C3c and C3d, was also tested to determine the site of the ISG65 interaction.

Binding impaired mutants of ISG65 and C3d were subjected to kinetic binding analysis using SPR. For interrogation of ISG65 mutants. His-C3d-Avi was used as ligand, and for C3d mutants. His-ISG₆₅₁₈₋₃₆₃-Avi was the ligand. Site-specific Avi-tag biotinylation for immobilisation on the SPR chip was carried out as previously described (doi: 10.1007/978-1-4939-2272-905 7_12 (2015)). SPR experiments were performed at 25° C. on BIAcore T200 using Series S sensor chip CAP (Cytiva). All binding analyses as well as dilutions were performed in SPR running buffer (20 mM HEPES pH7.5, 150 mM NaCl. 3 mM EDTA. 0.005% (v/v) TWEEN-20). Biotinylated ligands were coupled to flow path 2 at 10 μl·min⁻¹ for 120 s. Dilution series (1:1) of both analytes (ISG65, 1.5-100 nM: C3d. 0.78-50 nM) were applied to flow paths 1 and 2 at 30 μl·min⁻¹ for 120 s, followed by 300 s of dissociation time. The chip surface was regenerated in between titrations with 6 M guanidinium hydrochloride dissolved in 0.25 M sodium hydroxide. The binding data was reference subtracted, and kinetic and steady state affinity parameters were evaluated using BIAcore T200 evaluation software (GE Healthcare). Binding analyses of other fragments were carried out in an analogous manner.

ISG65 has indeed clear preferences for certain C3 activation fragments. Native C3 (KD=130 nM), the most abundant C3 species in human blood, has the lowest binding affinity, followed by C3b (KD=81 nM) and C3 (H₂O) (KD=18 nM). The high affinity for C3 (H₂O), the initiator of the AP, is likely to be a response to its low abundance in serum. The interaction with C3d (the proteolytically liberated thioester domain, TED) that takes place at even higher affinity (KD=7 nM), might be due to unimpeded movement in absence of the remaining C3 scaffold (i.e., C3c) (FIGS. 2A to 2E).

TABLE 1
Binding affinities between ISG65 and complement factors measured by surface plasmon
resonance. All data interaction parameters were calculated using the BIAcore T200 evaluation
software. For each interaction, the method of K_{D} calculation is indicated.

| | | | | | | | | |
|---|---|---|---|---|---|---|---|---|
| | | | | | | | | C3d |
| | | | kₒₙ₁ | kₒₙ₂ | k_{off1} | k_{off2} | K_{D} | binding |
| Ligand | Analyt | Mutation | (M⁻¹s⁻¹) | (M⁻¹s⁻¹) | (s⁻¹) | (s⁻¹) | (nM) | (%) |

Binding impaired mutants

| C3d-Avi | ISG65 | Wt | 1003000 | 0.90 | 0.04 | 0.002 | 27.9 | 100 |
|---|---|---|---|---|---|---|---|---|
| | | Y70A | 118000 | 0.008 | 0.14 | 0.58 | 78.2 | 36 |
| | | W211A | 320000 | 0.003 | 0.16 | 0.39 | 66.8 | 42 |
| | | Y293A | — | — | — | — | NB | NB |
| | | T297A | 171000 | 0.001 | 0.05 | 0.002 | 181.8 | 15 |
| | | | | | | | | ISG65 |
| | | | | | | | | binding |
| | | | | | | | | (%) |
| ISG65-Avi | C3d | Wt | 5241000 | — | 0.03785 | — | 7.2 | 100 |
| | | L1109A | — | — | — | — | 52.9 | 45 |
| | | E1110A | — | — | — | — | 15.7 | 8 |
| | | N1134A | 1893000 | — | 0.05419 | — | 28.6 | 25 |

C3 fragments

| | | | | | | | |
|---|---|---|---|---|---|---|---|
| ISG65-Avi | C3 | 941500 | — | 0.01698 | — | 18.0 | — |
| | (H₂O) | | | | | | |
| | C3b | 262200 | — | 0.02120 | — | 80.9 | — |
| | C3 | 81840.00 | 0.002427 | 0.05433 | 0.0005864 | 129.2 | |
| | C3c | — | — | — | — | NB | — |
| | C3a | — | — | — | — | 18 100 | — |

NB—no binding.

### Haemolytic Assays

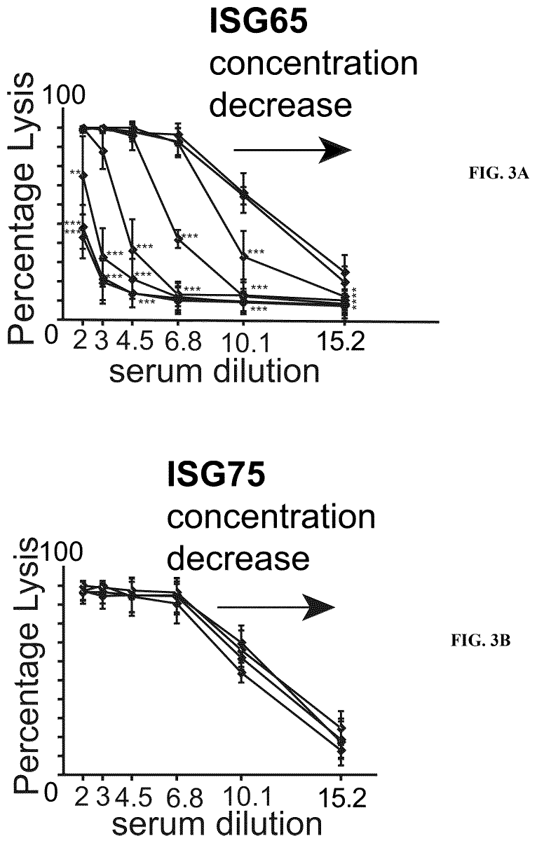

C3 and its functionally active fragment C3b are the central components of the alternative pathway (AP). To determine that IGS65 has an immune-regulatory role, a commercially available haemolytic assay (AP50 test, Haemoscan) for assessing the complement activity in human serum by quantification of complement mediated lysis of rabbit erythrocytes was used according to the manufacturer's instructions. Dilutions (2-15.2-fold) of non-heat inactivated human serum (Sigma-Aldrich) were incubated with serially diluted ISG65 and ISG75 (7.5-0 μM). Complement inhibition was determined from the extent of erythrocyte lysis measured at 415 nm. The concentration of free haemoglobin is hereby directly proportional to complement activity. The measurements were performed in triplicates (n=3). Statistical significance was calculated using two-way ANOVA in GraphPad Prism. The addition of ISG65 to the human serum resulted in a concentration-dependent decrease in haemolysis, indicating an inhibition of the AP (FIGS. 3A and 3B). To test for the specificity of these results, the experiment was repeated using another member of the T, brucei invariant surface glycoprotein family, ISG75 (FIGS. 3A and 3B). Increasing concentrations of ISG75 had no measurable effect on haemolysis, thereby confirming an ISG65-dependent inhibition of the complement cascade in vitro.

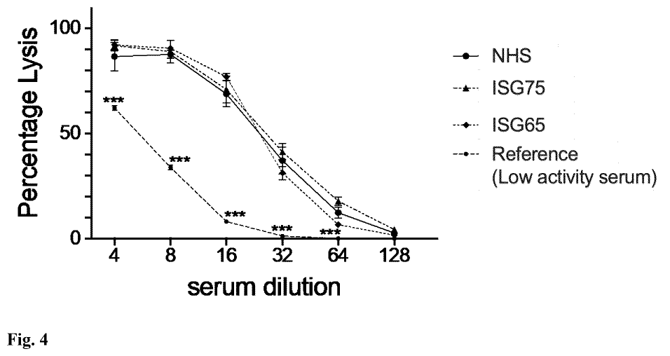

C3b also plays a role in the classical/lectin pathway (CP/LP) as a component of the CP/LP C5 convertase C4bC2aC3b. To investigate a potential regularory role of ISG65 in inhibition of the CP/LP via the CP/LP C5 convertase, another functional haemolytic assay (CH50 test, Haemoscan) was carried out (FIG. 4). Analog to the AP50 test, the CH50 was used to assess complement activity in human serum by quantification of complement-mediated lysis of sensitized sheep erythrocytes. No significant reduction of haemolyis could be measured upon addition of ISG65 at 24 μM concentration to serially diluted serum (FIG. 4). Dilutions of non-heat inactivated serum (Sigma-Aldrich) were prepared according manufacturer's instructions (Haemoscan). As in the AP50 test, ISG75 and also non-supplemented normal human serum (NHS) were used as a negative controls. Human serum with low complement activity served as a reference showing reduced complement-mediated lysis of erythrocytes. In summary, the functional haemolytic assays described here estbalished that ISG65 restricts the alternative pathway but not the classical pathway. Inhibition of the lectin pathway was not shown directly, but is implausible since CP and LP share the same C3b-containing component. C4bC2aC3b.

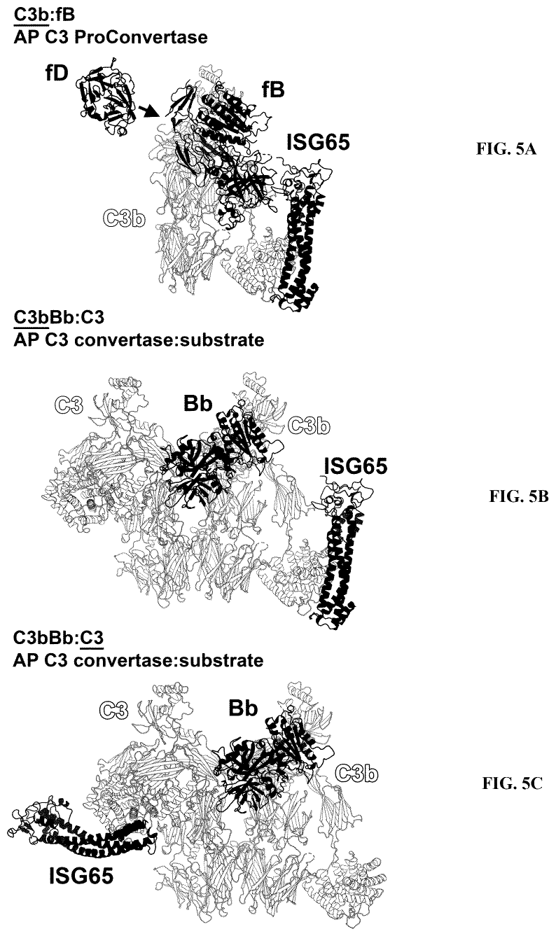

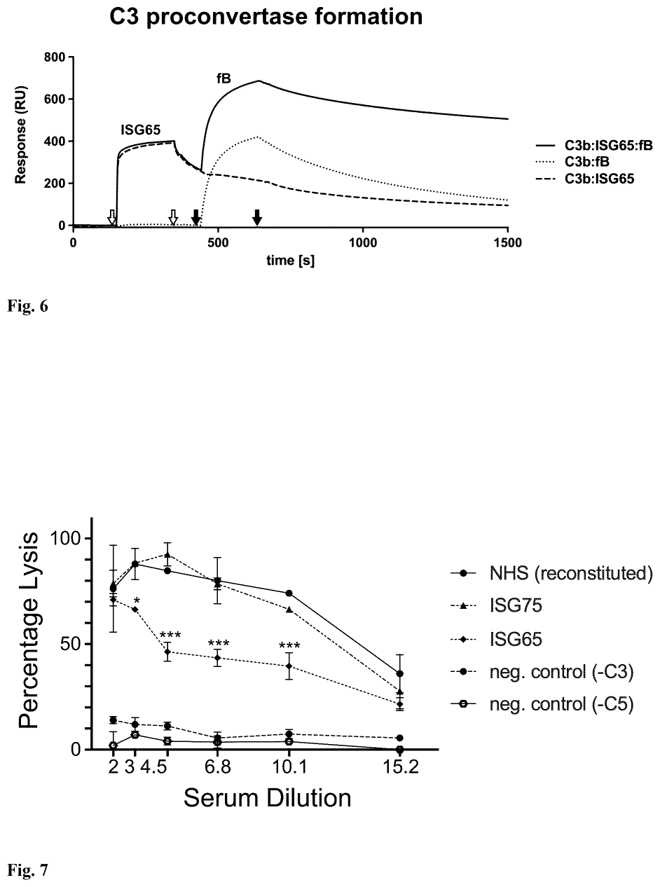

ISG65 does not prevent formation of the AP C3 proconvertase C3bBb and its binding to C3 substrate. We could show this by superimposing the structures of the AP C3 (pro) convertase in absence and presence of bound C3 substrate with the complex structures of ISG65: C3b and ISG65: C3 that are described here (FIGS. 5A to 5C). No clashes between ISG65 and components of the convertase were identified implying that ISG65 binding to C3b and native C3 does not prevent its assembly and substrate binding. The assembly of AP C3 proconvertase in presence of ISG65 was further confirmed by SPR (FIG. 6). C3b was hereby immobilised onto an SPR chip and ISG65 and factor B were subsequently injected. In a control experiment only factor B was injected. Both experiments gave rise to sensorgrams of the same height, unequivocally showing that ISG65 does not impair the binding of factor B to C3b.

Since ISG65 does not interfere with the AP C3 convertase we conclude that inhibition via binding to C3b must take place at the stage of the AP C5 convertase C3bBbC3b either by preventing assembly of C3bBbC3b via binding to one or both C3b molecules or by diminishing the ability of the assembled convertase to cleave C5 substrate. To further investigate how exactly ISG65 interferes with AP C5 convertase activity in vitro, we set out to separate convertase deposition on the cell surface from assembly of the membrane attack complex (MAC) in the terminal pathway which eventually results in hemolysis. To this end, the AP50 test (Haemoscan) was used, applying an adapted version of the manufacturer's instructions (FIG. 7). Rabbit erythrocytes were incubated with dilutions (2-15.2-fold) of Human complement C5-deficient serum (Sigma-Aldrich) which allowed for C5 convertase assembly on the cell surface, but stalled progression of the cascade due to a lack of C5 substrate. After incubation for 30 min at 37° C., erythrocytes were quickly washed to remove unbound material. The washing step, which was performed twice included centrifugation (400×g, 10 min, RT), the removal of the supernatant, and resuspension in dilution buffer (HaemoScan) before ISG65 and ISG75 (negative control) at 7.5 μM were added together with dilutions (2-15.2-fold) of Human Complement factor C3-deficient serum (Sigma-Aldrich). This step added back C5 substrate, but no C3 (as competitor). Erythrocyte lysis was performed for 30 min at 37° C. The modified AP50 test protocol was validated by efficient lysis of erythrocytes when both deficient sera were consecutively used (reconstituted NHS). Exchange of either serum with buffer prevented lysis. Addition of ISG65 to assembled AP C5 convertase revealed a significant effect on progression of the terminal pathway (FIG. 7). In comparison to reconstituted NHS, up to 40% less haemolysis was observed in presence of ISG65, but not in presence of ISG75. This showed that binding of ISG65 to pre-assembled AP C5 convertase can at least partially abrogate cleavage of C5 substrate in vitro (FIG. 7). This effect is however less pronounced than the one observed when ISG65 is incubated with NHS (at the same dilution factors) before addition of erythrocytes (standard AP50 test, FIG. 3A), which allows for both inhibition of assembly and substrate cleavage. Here, 90% less haemolysis was observed (FIG. 3A). We therefore conclude that, in vitro, ISG65 inhibits the activity of the AP C5 convertase through interfereing with both assembly and cleavage of C5 substrate.

Cryo-Electron Microscopy (Cryo-EM) Structures of ISG65 in Complex with (3 and (3b:

### Grid Preparation and Data Collection

### ISG65:3

3 μl (0.16 mg/ml) of ISG65: C3 were applied to freshly glow-discharged copper C/Flat 1.2/1.3 300 mesh grids (Protochips) and vitrified by being plunged into liquid ethane using a ThermoFisher Scientific Vitrobot Mark IV system (4° C., 100% relative humidity, no wait time, 2 s blotting time).

The grids were then transferred to a Titan Krios G3i microscope (ThermoFisher Scientific) equipped with a K3 detector (GATAN Inc.) and operated at 300 kV. Images were recorded at a magnification of 105000× while tilting the stage by 25°, yielding a pixel size of 0.86 Å. Multi-frame movies (40 frames with total dose of 41.10 e⁻/A²) were recorded using a nominal defocus (dF) range of −2.5 to −1.5 μm. Data were collected using the automated data collection software EPU (ThermoFisher Scientific).

### Isg65: C3b

To prepare grids used in the collection of datasets at a 25° tilt, 3.5 μl (0.1 mg/ml) of ISG65: C3b complex was applied to glow-discharged copper C/Flat, 300 mesh 1.2/1.3, and 2/2 TEM grids (Protochips). The grids were vitrified by being plunged into liquid ethane using ThermoScientific Vitrobot Mark IV system (4° C., 100% rel. humidity, 30 s waiting time, 4 s blotting time) and then transferred to a Titan Krios microscope (ThermoFisher Scientific) for data acquisition. For data collections at a 0° stage tilt, Au, 300 mesh, R1.2/1.3 TEM grids (Protochips) were coated with a graphene monolayer using an in-house developed protocol. 3.5 μl (0.15 mg/ml) of ISG65: C3b complex was applied to freshly plasma-cleaned TEM grids and vitrified as explained for grids used to obtain the 25° tilt dataset. The grids were subsequently transferred to a Titan Krios microscope (ThermoFisher Scientific) for data acquisition.

The data were collected at 300 kV using SerialEM (doi: 10.1016/j.jsb.2005.07.007 (2005)). The data were collected on a K2 direct electron detection camera positioned behind a Gatan Imaging Filter (Bioquantum 967, Gatan). The camera was operated in the electron counting mode, and the data were collected at the calibrated pixel size of 0.818 Å/px. The data from a 5.0 s exposure were split into 40 frames comprising an overall dose of 60 c/Å².

### Image Processing

### ISG65: C3

A total of 10,380 movies were acquired during data collection. Motion correction as well as CTF correction (CTFFIND4 wrapper) were done using cryoSPARC (doi: 10.1038/nmeth.4169). 5,508,854 particles were picked using the reference-free Gaussian Blob picker in cryoSPARC. Particles were extracted with a 400 px box with 2-fold binning applied which resulted in a pixel size of 1.72 Å/px. Iterative panning using reference free 2D classification in cryoSPARC was performed until a subset of 432,224 particles was identified. The particles were re-extracted using a 400 px box with no binning applied. The re-extracted particles were submitted to another round of reference free 2D classification in Relion 3.1 (doi: 10.7554/eLife.42166). 406,545 particles were selected and used for ab initio modelling and subsequently subjected to the 3D classification job in Relion. Particles were classified into 5 classes, all yielding well defined reconstructions of the desired complex. All particles selected after 2D classification in Relion were later used for creation of an ab initio model in cryoSPARC, which was subsequently refined using the non-uniform refinement procedure (cryoSPARC v3.2 and later) and local CTF refinement to yield the final reconstruction.

### ISG65: C3b

A total of 18,062 movies were collected at a 25° stage tilt in two separate data collections (for two different grid types), and 8960 movies were collected at a 0° stage tilt on graphene coated grids. The processing of each dataset was performed independently before merging selected particles with previous data collections.

Motion correction and CTF correction for all 27,022 movies were performed using cryoSPARC's patch motion and patch CTF correction implementations, respectively.

On images acquired at a 25° stage tilt, particles were picked using the convolutional neural network-based particle picker, crYOLO (doi: 10.1038/s42003-019-1191 0437-z). Particles were initially identified using the supplied general model. The resulting particle picks were manually inspected, and reference free 2D classification was performed in cryoSPARC. Particles constituting the 2D classes which represented the protein (regardless of whether they were the desired complex or not) were selected and used to re-train the outer layers of the general model on a subset of micrographs at varying defocus, crYOLO identified a total of 2,836,445 particles (U.S. Pat. Nos. 1,414,868 and 1,421,577). The processing of the two datasets followed the same overall processing pipeline, using cryoSPARC. Particles were extracted using a box with 400 px and two-fold binning, resulting in a pixel size of 1.656 Å/px.

Iterative, reference-free 2D classification was performed until satisfactory 2D classes were identified. The selected particles were subjected to ab initio modelling using 3 classes. Models representative of the desired ISG65: C3b complex and C3b were chosen for subsequent Heterogenous Refinement. Particles constituting the reconstruction representing ISG65: C3b were selected, re-extracted using a 400 px box (no binning applied resulted in a pixel size of 0.828 Å/px), and subjected to Non-uniform Refinement.

For images acquired at a 0° stage tilt, the gaussian particle picker in cryoSPARC was utilised to identify particles. The remaining processing was performed as described for data collected at a 25° stage tilt.

For the final reconstruction, 204,946 combined particles were re-subjected to ab initio modelling using 2 classes. The class representative of the desired complex was subjected to Non-uniform Refinement and local CTF refinement.

A total of 145,172 particles, of which 54,453 particles came from datasets at 25° tilt (22,266 and 32,187, respectively) and 90,719 particles came from a 0° tilt dataset, contributed to the final reconstruction.

### Model Building and Refinement

### ISG65: C3

The initial step in model building was performed by docking starting models (the crystal structure of C3 [PDB 2 Å⁷³] and an ISG65 model predicted by AlphaFold2 (doi.org/10.1038/s41586-021-03819-2)) into the obtained electron density map using Phenix (doi: 10.1107/S2059798319011471). After an initial real space refinement in Phenix, both models were independently and iteratively refined using a combination of manual refinements in Coot as well as automated real-space refinements using Phenix and REFMAC5 (doi: 10.1107/S0907444911001314). Model validation was performed using MolProbity (doi: 10.1093/nar/gkm216).

After the models had individually reached satisfactory validation metrics and map correlation, they were manually and automatically refined together using Coot (doi: 10.1107/S0907444904019158) and Phenix (doi: 10.1107/S2059798319011471) to model interactions between the two proteins.

### ISG65: C3b

Due to large variations in local resolution of the obtained electron density map, the modelling of ISG65 at atomic resolution was not possible. Therefore, the atomic model obtained from the ISG65: C3 data was simply docked in the electron density map using Phenix. For modelling C3b, a starting model (the crystal structure of C3b [PDB 2107]) was docked in the obtained electron density via Phenix. After an initial real space refinement in Phenix, the model was iteratively refined using a combination of manual refinements in Coot as well as automated real-space refinements using Phenix and REFMAC5. Model validation was performed using MolProbity.

### Structure Description

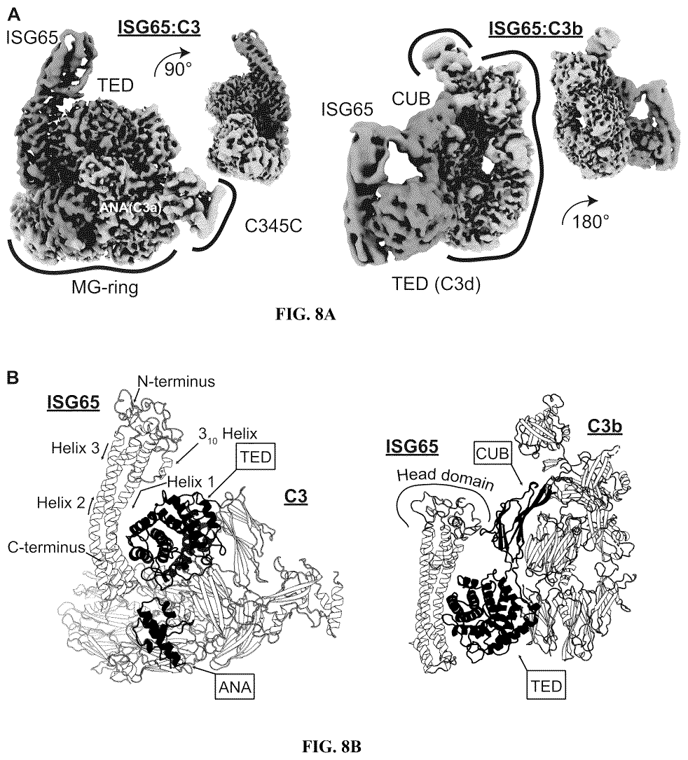

The ISG65: C3 complex could be reconstructed to a reported FSC 0.143 of 3.58 A, while ISG65: C3b was reconstructed to FSC 0.143 of 3.59 A. However, the local resolution range across the reconstructions varies significantly, especially for ISG65: C3b. The resolution of the reconstruction corresponding to the rather rigid MG-ring is in the near-atomic range of 3.5 A. The local resolution in the CUB and TED region varies between 4 and 5.5 A, while at the interface and in the remainder of ISG65, it ranges from 5 to beyond 10 A. Secondary structure features like β-sheets in the CUB domain and α-helices in ISG65, however, could still be identified (FIG. 8A). For ISG65: C3, the local resolution predominantly ranges from 3 to 5 A, with lower resolutions in the disordered head region of ISG65, the flexible C345c domain, and the glycan trees in the two glycosylation sites. The higher variance in local resolution across the ISG65: C3b reconstruction is likely caused by an increased flexibility of the extended domains CUB and TED relative to the MG-ring. In contrast, C3 adopts a more compact conformation with TED wedged between the MG-ring and ANA, resulting in reduced flexibility and thus variance within the molecule. This is also reflected in the sloped shape of the FSC of ISG65: C3b, and a rather vertical drop of the FSC in the ISG65: C3 reconstruction.

### ISG65:03b

Despite large variations in the local resolution across the reconstruction, C3b could be modelled except for the flexible regions Asn93-Gly 101, Gln312-Leu314, Pro665-Ala667, Ser749-Leu751. Glu1372-Ala1380, and Ser1523-Asp1524. C3b resembles previously published structures (e.g., PDB 2107). Minor re-arrangements of the TED and the CUB domain, especially in residues Gln1161-Pro1287, can be observed in C3b and may be caused by ISG65 binding (data not shown).

The lack of near-atomic resolution in ISG65 of the ISG65: C3b reconstruction prevented us from drawing conclusions about details of the binding interface. However, the superposition of the atomic model of ISG65 with its counterpart in the C3 complex suggests an identical binding interface with TED. In addition to the TED interface, the electron density corresponding to the N-terminal ‘head’ region of ISG65 also suggests contacts to the CUB domain on C3b (FIG. 8A, right). Should this interaction occur in a physiological context, the second attachment point could lock the TED domain in a conformation that further restricts its free movement. This lock would thereby also prevent the movement of the hydrolysed thioester bond of Gln 1013 towards the membrane.

### ISG65:03

The near atomic resolution of the ISG65: C3 reconstruction allowed us to build the first reported model of a T. b. gambiense Invariant Surface Glycoprotein and to describe its interaction with human complement C3 on a structural basis.

ISG65 adopts a three-helix bundle fold (Helix 1, Lys36-Lys84: Helix 2, Asp 100-198 Asn144; Helix 4, Thr259-Glu300) with a 20° curvature along its longest axis. The disordered N-terminal residues Leu18-Leu34 as well as 200 residues Arg146-Ser202, and Lys227-Met255 constitute intrinsically disordered 201 loops and thus were not resolved. Instead of distinct conformations, a cloud of electron density was observed, describing the average volume occupied by the loops. Using MS-based disulphide mapping, we were able to identify three disulphide bridges in the disordered ‘head’ region of ISG65 (Cys12-Cys137, Cys149-Cys168, and Cys209-Cys220). The atomic model of the disordered regions was predicted using iterative, template-guided modelling in AlphaFold2 using real space constraints from the reconstruction and disulphide bonding as selection criteria for the models. Although resolved at a lower local resolution, the loop connecting helix 1 and helix 2 (Lys85-Asp100) close to the C-terminus could be modelled. Due to a well resolved electron density, we could confidently place the short helix 3 (residues Gln217 to Val226) and the preceding loop (Asp210-Leu216) near the ‘head’ region of ISG65. Residues Thr203-Asp210 were not resolved to atomic resolution but appear to have a 310 helical conformation. At the C-terminal end of the model, residues Gln301-Leu305 form a loop connecting helix 4 with the short helix 5 (Thr306-Ala312), which notably points towards the N-terminus of the protein, away from the parasite surface. Beyond these residues, only poorly resolved electron density could be observed which made accurate placement of residues in this region impossible. With the exception of the disordered regions Ala666-Ser672. Arg740-Leu751, and Glu 1634-Asn1642. C3 was well resolved and could be modelled in its entirety (FIG. 8B). Positioned on top of the ANA domain (C3a, when released), binding of ISG65 to C3 is predominantly mediated via multiple hydrophobic contacts and four distinct hydrogen bonds with the thioester domain (Arg 77_{ISG65}-Leu 1 109_{C3}) and three hydrogen bonds from Tyr293 (Tyr293_{ISG65}-Glu1110_{C3}. Tyr293_{ISG65}-Glu111_{C3} and Tyr293_{ISG65}-Glu1119_{C3}). The interaction between the proteins is further facilitated by the almost perfect complementary fit between the convex tip of TED and the concave 3-helix-bundle of ISG65, a likely result of molecular co-evolution (FIG. 8B).

### Validation of the Interface

In order to dissect the binding interface into segments and key residues critically contributing to the overall binding affinity, we mutated surface-exposed, non-structurally relevant residues to alanines. These key residues were chosen at the upper, middle, and lower end of the binding site in ISG65 as well as at the convex tip of TED, based on the higher resolution structure of ISG65: C3. The binding affinities of recombinantly expressed C3d mutants to ISG65, and vice versa. ISG65 mutants to C3d (representing TED in C3), were determined by SPR and compared against the affinities of the wild-type (wt) proteins. Correct protein folding of the mutants was assessed via circular dichroism spectroscopy (CDS). ISG65 mutations along the entire length of the interface contributed to the binding as indicated by the decrease in dissociation constants (K_{D}s) (Table 1). Mutation of Tyr293 completely abrogated ISG65 binding to C3d. As Tyr293 is one of the residues that contributes hydrogen bonds to the interaction with C3d and engages in several hydrophobic contacts with C3d residues as well, a severe reduction in binding affinity was to be expected. Notably, mutation of Trp211 caused a decrease in binding affinity to 42%. This is in agreement with the well-resolved electron density of the adjacent loop (Asp210-Leu216) as well as the side chain of Trp211. Although only contributing two hydrophobic contacts, this interaction might be crucial for correct alignment of C3d at the upper end of the interface. Similarly, mutation of two of the four hydrogen bond forming residues on C3d. Leu1109 and Glu1011 leads to a notable decrease in binding affinity. This can also be attributed to the fact that the side chains of Leu1 109 and Glu1110 are located at the core of the interface and engage in multiple hydrophobic contacts. Despite its localisation outside the interface in the ISG65: C3 structure, the mutation of Arg1134 on C3d caused a decrease in binding affinity to 24%. To aid the identification of potential key mediators of the interaction, we also employed hydrogen-deuterium exchange mass spectrometry (HDX-MS). Here. Arg1134 was centred in a ‘protection hotspot, indicating limited solvent accessibility. When taken into consideration that this residue is located on a long (26 aa) loop close to the interface, it seems conceivable that a transient interaction with ISG65 binding may occur.

ISG65 interacts with ANA C3a

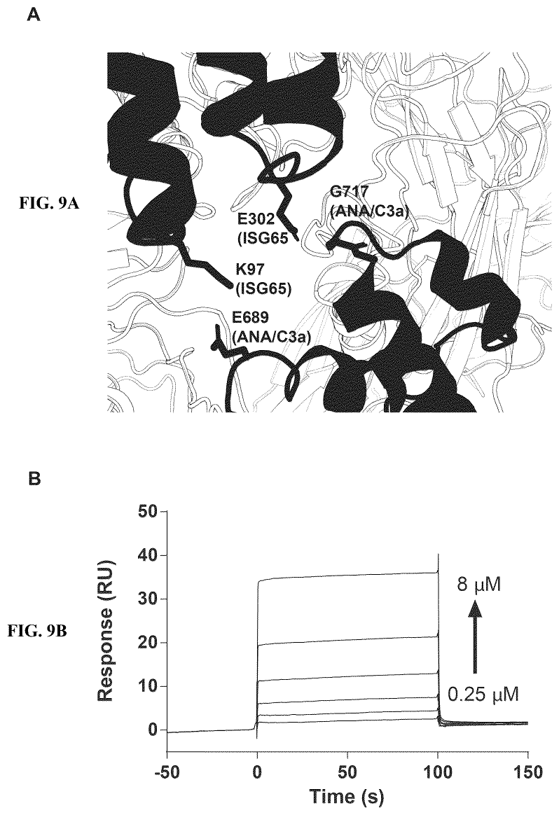

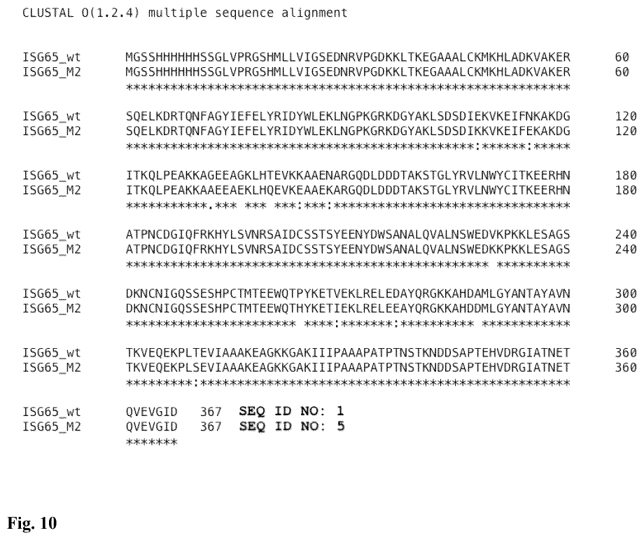

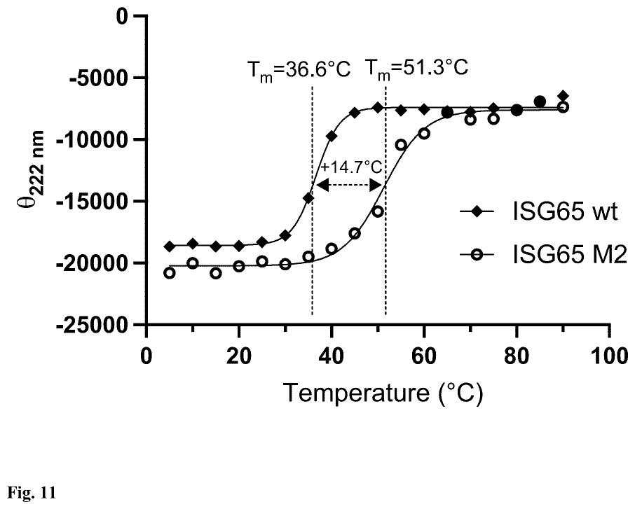

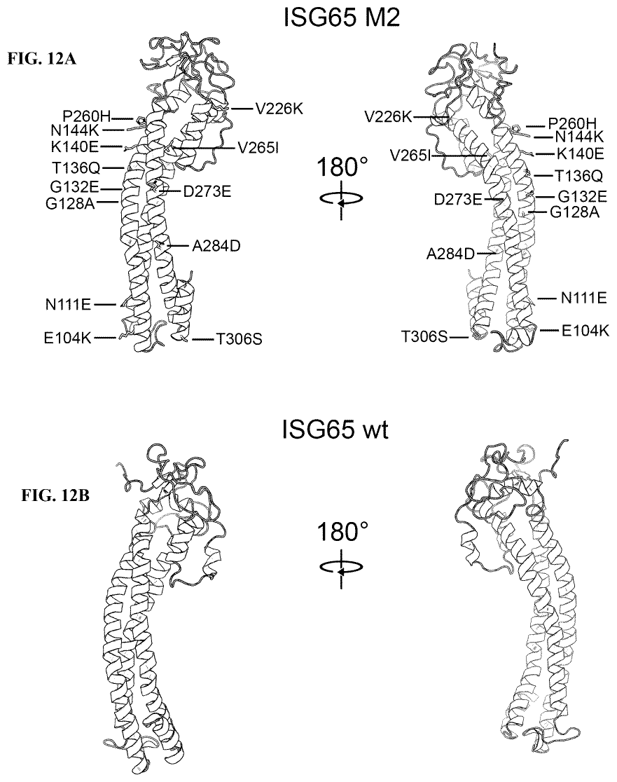

In the ISG65: C3 structure, a second, smaller interface is formed between the membrane proximal, C-terminal end of ISG65 and the ANA domain. Within the interface, we could identify two possible hydrogen bonds, Lys97_{ISG65}-Glu689_{C3} and Glu302_{ISG65}-Gly717_{C3} (FIG. 9A). Although the reconstruction in this region suffers from poor electron density for the side chains, the identified residues are well within bonding distance. Furthermore, all contributing residues are located in loops of varying lengths and thus are potentially subject to large degrees of freedom, facilitating transient interactions. To investigate whether the interface between ANA and ISG65 may be physiologically relevant, we confirmed the specific binding of recombinant C3a to ISG65 by SPR (FIG. 9B, Table 1). In agreement with the lack of clear electron density, kinetic profiles revealed high on- and off-rates which is indeed indicative of a rather short-lived interaction between ISG65 and free C3a.

### Cloning, Expression and Purification of ISG65 M2

A DNA fragment (Genewiz) encoding amino acids (aa) 18-363 of ISG65 from T. b. gambiense (Tbg.972.2.1600) including ISG65 M2 specific mutations was codon-optimised for bacterial expression and cloned into the pET15b plasmid using Gibson assembly method (New England Biolabs). For recombinant expression in E, coli T7 shuffle cells (New England Biolabs), a plasmid encoding an N-terminal hexa-histidine-tag (His-ISG₆₅₁₈₋₃₆₃) was generated. ISG65 M2 was produced overnight at 22° C., after induction of protein expression with 1 mM Isopropyl-b-D-thiogalacto-pyranoside (IPTG). Soluble protein was purified from bacterial lysate by immobilised metal-affinity chromatography (IMAC) using nickel-nitrilotriacetic acid (NTA) beads (Qiagen) and gravity-flow columns (Bio-Rad). After IMAC, the purified proteins were concentrated using Amicon Ultra centrifugal filters (Merck Millipore) and subjected to size-exclusion chromatography using a Superdex 200 Increase 10/300 GL (GE Healthcare) equilibrated with 20 mM HEPES (pH 7.5) and 150 mM NaCl.

The DNA sequence of the expression construct was SEQ ID NO: 15:

ATGGGCAGCAGCCATCATCATCATCATCACAGCAGCGGCCTGGTGCCGC
GCGGCAGCCATATGCTGCTGGTGATTGGCAGCGAAGATAACCGCGTGCC
GGGCGATAAAAAACTGACCAAAGAAGGCGCGGCGGCGCTGTGCAAAATG
AAACATCTGGCGGATAAAGTGGCGAAAGAACGCAGCCAAGAACTGAAAG
ATCGCACGCAGAACTTTGCGGGCTATATTGAATTTGAACTGTATCGCAT
TGATTATTGGCTGGAAAAACTGAACGGCCCGAAAGGCCGCAAAGATGGC
TATGCGAAACTGAGCGATAGCGATATTAAAAAAGTGAAAGAAATTTTTG
AAAAAGCGAAAGATGGCATTACCAAACAGCTGCCGGAAGCGAAAAAAGC
GGCGGAAGAAGCGGAAAAACTGCATCAAGAAGTGAAAGAAGCGGCGGAA
AAAGCGCGCGGCCAAGATCTGGATGATGATACCGCGAAAAGCACCGGCC
TGTATCGCGTGCTGAACTGGTATTGCATTACCAAAGAAGAACGCCATAA
CGCGACCCCGAACTGCGATGGCATTCAGTTTCGCAAACATTATCTGAGC
GTGAACCGCAGCGCGATTGATTGCAGCAGCACGAGCTATGAAGAAAACT
ATGATTGGAGCGCGAACGCGCTGCAAGTGGCGCTGAACAGCTGGGAAGA
TAAAAAACCGAAAAAACTGGAAAGCGCGGGCAGCGATAAAAACTGCAAC
ATTGGTCAGAGCAGCGAAAGCCATCCGTGCACCATGACCGAAGAATGGC
AGACCCATTATAAAGAAACCATTGAAAAACTGCGCGAACTGGAAGAAGC
GTATCAGCGCGGCAAAAAAGCGCATGATGATATGCTGGGCTATGCGAAC
ACCGCGTATGCGGTGAACACCAAAGTGGAACAAGAAAAACCGCTGAGCG
AAGTGATTGCGGCGGCGAAAGAAGCGGGCAAAAAAGGCGCGAAAATTAT
TATTCCGGCGGCGGCGCCGGCGACCCCGACCAACAGCACCAAAAACGAT
GATAGCGCGCCGACCGAACATGTGGATCGCGGCATTGCGACCAACGAAA
CCCAAGTGGAAGTGGGCATTGATTAA

The translated sequence, including the affinity tag, was SEQ ID NO: 16:

MGSSHHHHHHSSGLVPRGSHMLLVIGSEDNRVPGDKKLTKEGAAALCKM
KHLADKVAKERSQELKDRTQNFAGYIEFELYRIDYWLEKLNGPKGRKDG
YAKLSDSDIKKVKEIFEKAKDGITKQLPEAKKAAEEAEKLHQEVKEAAE
KARGQDLDDDTAKSTGLYRVLNWYCITKEERHNATPNCDGIQFRKHYLS
VNRSAIDCSSTSYEENYDWSANALQVALNSWEDKKPKKLESAGSDKNCN
IGQSSESHPCTMTEEWQTHYKETIEKLRELEEAYQRGKKAHDDMLGYAN
TAYAVNTKVEQEKPLSEVIAAAKEAGKKGAKIIIPAAAPATPTNSTKND
DSAPTEHVDRGIATNETQVEVGID

ISG65 M2 binds to C3b

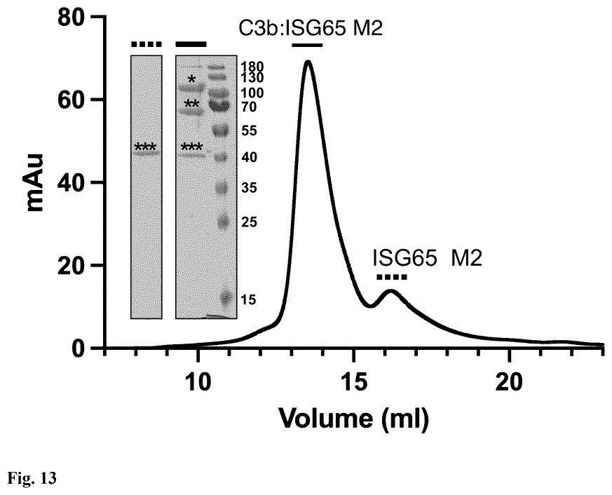

All thermostability increasing mutations in ISG65 M2 were introduced outside the C3b binding interfaces. In order to confirm C3b binding in solution, analytical size exclusion chromatography was carried out (FIG. 13). ISG65 M2 was hereby mixed and incubated with C3b at 3-fold molar excess, concentrated, injected onto a gel-filtration column (Superdex 200 10/300) and the eluate fractionated. Analysis of protein fractions underneath the main UV peaks by SDS-PAGE revealed that free ISG65 M2 has been depleted and elutes earlier, together with C3b, indicating a specific, high-affinity interaction between both proteins (FIG. 13).

### Circular Dichroism Spectroscopy

In order to compare the differences in melting temperatures (Tm) between ISG65 wt and ISG65 M2, far-UV CD experiments were carried out on a Jasco J-1500 spectropolarimeter with a 0.2 mm path cell. ISG65 was dissolved in 10 mM HEPES pH 7.5, 150 mM NaF at a concentration of 0.4 mg/ml. Spectra were recorded between 195 and 260 nm wavelength at an acquisition speed of 10 nm/min and corrected for buffer absorption. For calculation of melting curves at 222 nm, spectra were recorded every 5 degrees between 5° C., and 80° C., with a slope of 0.16° C./min. During measurements, the temperature was kept constant. The raw CD data (ellipticity 0 in mdeg) were normalized for the protein concentration and for the number of residues, according to the equation below, yielding the mean residue ellipticity ([0] in deg cm² mol-1), where MM, n, C, and/denote the molecular mass (Da), the number of amino acids, the concentration (mg/mL), and the cuvette path length (cm), respectively (FIG. 11).

$$[ θ ] = \frac{θ \cdot MM}{n \cdot C \cdot l}$$

Acute Toxicity Study of ISG65 and ISG65 M2 after Intraperitoneal Administration in Mice

### Methods

The acute toxicity study was carried out using C57BL/6 JRccHsd mice, at the beginning of the study 7-8 weeks old males, with body weight of 20.98g-24.98g. The mice were allocated into 9 groups of three mice each. 4 groups were treated with various concentrations of ISG65, 4 groups were treated with various concentrations of ISG65 M2, and one group was a control group.

The tested proteins, ISG65 and ISG65 M2 were administered to the animals at the dose 400 μg, 800 μg, 1200 μg and 2000 μg per 30 g of body weight. The proteins were administered intraperitoneally (i.p.) in PBS (phosphate buffer saline). The control group only received the vehicle (PBS). Daily Observations: All mice were observed for clinical signs, morbidity or mortality once a day during acclimation, frequently during the first 30 minutes after dosing and then 1 hour, 2 hours, 3 hours and 24 hours after the dosing and then daily in the morning and in the afternoon thereafter, for a total of 12 days (including the day of necropsy). Onset, duration and severity of any signs were recorded.

Clinical Observations included: Signs of toxicity, changes in the skin and fur, eyes and mucous membranes, respiratory, circulatory, autonomic and central nervous system, somatomotor activity and behavior pattern. Attention was directed to observations of tremors, convulsion, salivation, diarrhea, lethargy, sleep, coma and changes in gait, posture and response to handling, the presence of clonic or tonic movements and stereotypes.

Body Weight: All mice were individually weighed before randomization into the groups, before administration of the tested proteins (Day 1), on Day 4, on Day 8 and before necropsy (Day 12). Terminal Observations: All animals survived until their scheduled euthanasia (isoflurin anesthesia) on Day 12 and received a gross necropsy. All gross pathological changes were recorded.

Necropsy: All animals were weighted and externally examined. The external surface of the body and all orifices of the body were examined. The thoracic, visceral and cranial cavities were opened and examined macroscopically. Any abnormalities were recorded with details of location, color, shape and size.

Sample collection: In the control and the dose groups of both tested proteins, the livers, hearts and brains of all three animals in the each group were removed during necropsy and placed in a 4% buffered formaldehyde.

### Results

All the animals in the study were in good clinical condition during the acclimatization.

No clinical signs were observed in any of the groups of animals after the administration of the tested proteins or vehicle (control group) and during the observation period (Day 1-Day 12).

There were no significant changes in weight during the experiment in either the control or dose groups, and neither the beginning and end of the experiment.

A thorough necropsy was performed on all animals after euthanasia with inhaled isoflurane without exsanguination. Only animals from the highest dose groups (2000 μg/30 g) were exsanguinated by cardiac puncture under anaesthesia.

During necropsy of all animals except of animals of groups having received the dose 800 μg/30 g, spleens were removed and immediately cooled on ice. In addition, the brain, liver and heart were removed and placed in 4% formaldehyde.

All animals were in satisfactory nutritional condition. On examination, retroperitoneal fat was present in adequate amounts in all animals.

No pathological changes were detected in any of the animals of control group and almost all groups having received the tested proteins at the scheduled necropsy. The exception was the group having received ISG65 at the dose of 1200 μg/30 g where two animals had one slightly enlarged liver lobe. However, such finding did not occur in the group having received ISG65 at the dose of 2000 μg/30 g.

### Conclusions

The tested proteins showed no signs of acute toxicity in mice when administered intraperitoneally at doses of 400, 800, 1200, 2000 μg/30 g body weight under conditions used in this study.

### Asessmentment of Neutralising Antibody Generation Against ISG65

### Methods

Protein labelling: ISG65 was dialysed into 200 mM sodium bicarbonate buffer, 150 mM NaCl, pH 8.3 and subsequently concentrated to 8 mg/mL.

AF₅₉₄ NHS ester (Invitrogen) was resuspended to 10 mg/mL in anhydrous DMSO and added to a final concentration of 1 mg/mL. After 1h incubation at room temperature (RT), the reaction was quenched by addition of tris(hydroxymethyl)aminomethane to a final concentration of 200 mM. Free dye was removed by size exclusion chromatography (SEC) using a Superdex 200 10/300 Increase GL column (GE Healthcare), pre-equilibrated in HBS (20 mM HEPES, 150 mM NaCl, pH 7.5). Elution fractions containing ISG65_{AF594} were collected and pooled.

Protein complexes of ISG65_{AF594} with C3d and Fab (fragment antibody, derived from an ISG65 specific monoclonal antibody (mAb) that binds a linear epitope with the amino acid sequence TAKSTGL (SEQ ID NO: 18) within the head domain of ISG65), respectively, were obtained by concentrating ˜1 mg of either protein with 300 μg of ISG65_{AF594} to approx. 200 μL. Subsequently, excess protein was removed via SEC on a Superdex 200 10/300 Increase GL column (GE Healthcare), pre-equilibrated in HBS.

Elution fractions containing both ISG65_{AF594} and C3d/Fab were identified by SDS-PAGE, pooled and concentrated to ˜1 mg/mL before flash-freezing in liquid nitrogen. Samples were subsequently stored at −80° C. until use.

Preparation of mouse spleens and isolation of B-cells: The spleens from mice immunised with 40 μg of ISG65 per gram of body weight were harvested, fatty tissue removed and stored on ice. 1.5 hours after sacrifice of the animals, B-cells were isolated from spleens using a gentleMACS tissue dissociator (Miltenyi Biotec) in combination with the mouse spleen dissociation kit and C-tubes (Miltenyi Biotec), according to the manufacturer's instructions. Contaminating erythrocytes were lysed using ACK (Ammonium-Chloride-Potassium) lysis buffer.

Labelling and sorting of B-cells: The remaining B-cells were washed and resuspended in PBS for staining with Zombie NIR dye (BioLegend). After staining with the viability marker, the cells were washed and resuspended in ‘staining buffer’ (PBS, 1% FBS (v/v)). FcR Blocker (Miltenyi Biotec) was added to minimize unspecific labelling according to the manufacturer's instructions. After washing and resuspending the cells in staining buffer, anti-CD19-FITC (BioLegend (1:10 (v/v)) and ISG65_{AF594} (19 μg/mL final) were added and incubated for 30 mins in the dark, at RT. After the incubation period, the cells were washed and resuspended in staining buffer.

In order to improve the signal-to-noise ratio of the experiment, B-cells exhibiting binding to ISG65_{AF594} were pre-selected and isolated. To do so, positively labelled, vital B-cells singlets (Zombie NIR negative, CD19-FITC positive, ISG65_{AF594} positive) were gated for and sorted into RPMI-16 media using a CytoFlex cytometer (Beckmann), supplemented with 1% (v/v) low-endotoxin FBS. The final cell concentration was approximately 2×10\hat{ }6, cells/mL.

The cells were cultured over night at 37° C. The following day, the cells were harvested by centrifugation and washed three times with PBS.

After the final washing steps, the cells were re-suspended in 100 μL PBS. The cell suspension was split into 25 μL aliquots and 1 μL of PBS (negative control), ISG65_{AF594}, ISG65_{AF594}: C3d or ISG65_{AF594}: Fab were added (˜40 μg/mL final).

The samples were then subsequently analysed in context of AF₅₉₄-positive cells, using a CytoFlex cytometer. Sufficient washing was confirmed by absence of the anti-CD19-FITC signal (data not shown).

### Results

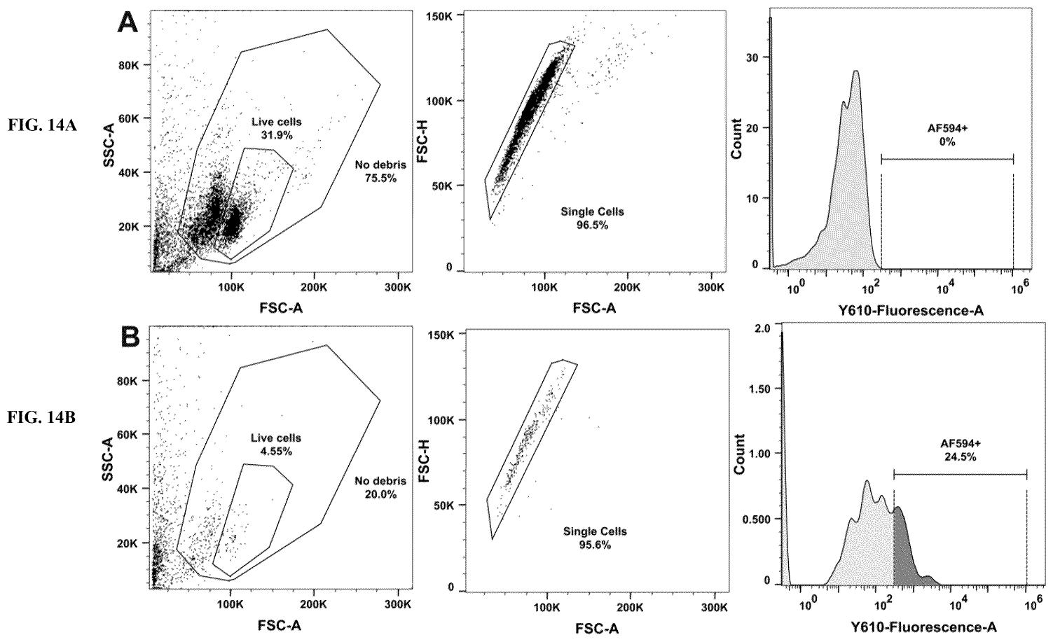

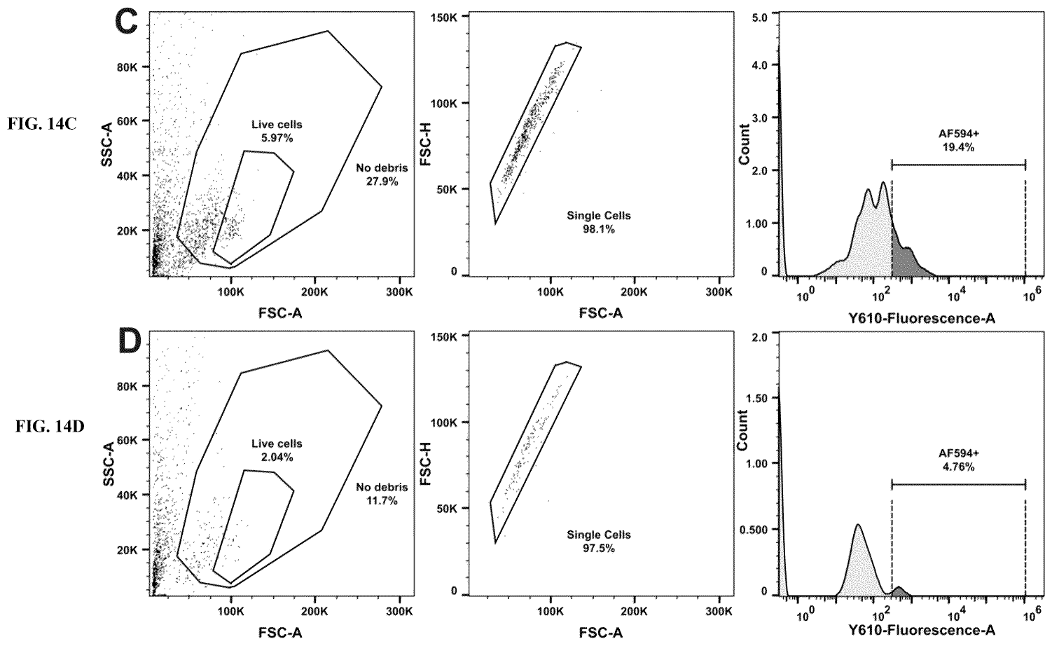

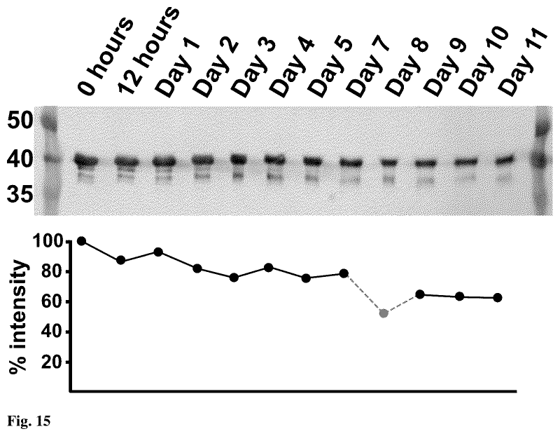

No remaining AF₅₉₄ positive cells could be detected in the negative control (FIG. 14A). Addition of ISG65_{AF594} alone resulted in 24.5% of AF₅₉₄ positive (+) B-cells (FIG. 14B). Addition of ISG65 with its natural binding partner C3d, the proteolytically cleaved thioester containing domain (TED) from the biologically active fragment C3b, caused no significant decrease in AF594 positive cells (19.4% AF594+) indicating that B-cell presented antibodies recognise different epitopes outside the C3b binding site (FIG. 14C).

However, addition of ISG65 in presence of a mAb-derived Fab that binds an epitope in the head domain of ISG65, causes a significant decrease in AF594 positive cells (4.76% AF594+) suggesting competition of B-cell presented antibodies for epitopes in the same region (FIG. 14D).

### Conclusions

These experiments indicate that antibodies raised against ISG65 upon immunization are non-neutralizing for the interaction with C3b and mainly targeting the loop-rich head domain of ISG65 near the N-terminus that acts as an immune decoy. The lack of neutralizing antibodies indicates that ISG65 can remain functional over an extended period of time in the circulatory system of mice.

### Long-Term Stability of ISG65 in Human Plasma

### Methods

Generation of mouse anti-ISG65 polyclonal serum: A standard CFA-IFA immunisation protocol was used to obtain polyclonal anti-serum in mice. In brief, female BALB/c mice were immunised on day 0 with a subcutaneous injection of 100 μg protein in complete Freud's adjuvant (Sigma-Aldrich) (100 μl protein+100 μl adjuvant), and on days 21, 42 and 62 with an intraperitoneal injection of 50 μg (100 μl protein+100 μl adjuvant) protein in incomplete Freud's adjuvant (Sigma-Aldrich). Mice were sacrificed on day 64 and the serum was isolated. The polyclonal serum was aliquoted, flash frozen in liquid nitrogen and stored at −80° C. until use.

Time course: 53 μL of ISG65 was added to 500 μL of human plasma to a final concentration of 2 mg/mL. After thorough mixing, the aliquot of the reference sample (Day 0) was taken immediately from the sample tube and diluted into PBS containing 0.05% Tween-20 (PBS-T, 1:20), before flash freezing in liquid nitrogen. Aliquots were taken over a time course of 11 days, each day at the same time. All frozen aliquots were stored at −80° C. until further use.

The sample tube, closed and sealed with parafilm, was constantly incubated at 37° C., throughout the remainder of the experiment. Before removing aliquots at the given time points, the sample tube was vortexed gently before briefly centrifuging the tube (<500×g) to collect the condensate from the lid.

The appropriate sample volume was removed and diluted into PBS-T (1:20), before flash freezing in liquid nitrogen. Aliquots for all following time-points (Day 1-Day 11) were acquired as described.

Western blotting: The frozen aliquots were thawed simultaneously in a heated water bath at 37° C. before any precipitate was removed by centrifugation (21.000×g, 4° C., 5 min). 12 μL of the cleared supernatant were combined with 3 μL of 6× non-reducing loading dye before being heated at 100° C. for one minute. The aliquots were centrifuged (21.000×g, RT, 3 min) before all 15 μL were being loaded onto a 12.5% polyacrylamid gel for SDS-PAGE (43 min, RT, 220V constant).

Proteins were transferred onto a Nitrocellulose membrane, pre-soaked in transfer buffer (20 mM Tris, 190 mM glycine, 20% Methanol, pH 8.3) using a Mini-Transfer cell (BioRad) for 1.5 hours (100 V constant, 4° C.).

The membrane was transferred into SuperBlock T20 (TBS) blocking buffer (Thermo Scientific) and blocked overnight at 4° C. After removing the blocking solution, the membrane was rinsed and washed three times for 5 minutes with PBS-T.

Subsequently, the membrane was incubated with polyclonal anti-ISG65 mouse serum (1:100, 2h, RT). After removing the polyclonal serum, the membrane was rinsed and washed three times as described previously, before incubation with the IRDye 680RD conjugated donkey anti-mouse IgG secondary antibody (LI-COR Biosciences) at a final concentration of 1:10.000 (1.5h, RT, dark). From this point onwards, the membrane was protected from light. After the incubation period, the membrane was washed three times, as described, before imaging.

Analysis Quantification: The Western Blot was imaged wet on an Odyssey CLx (LI-COR Biosciences) with the ImageStudio Software (Version 5.2.5). Fluorescence was detected using the 700 nm channel. The fluorescence intensity was analysed with the ImageJ software.

In brief, an area encompassing the entire reference band (0 hours, FIG. 15) was selected and brightness measured within. The same area-size and shape was used to measure the brightness of the other bands. For each lane, the background signal was determined above the measured protein band using the same area. The final intensity was obtained by subtracting the measured brightness of the background from the respective protein band. The percentage of fluorescence intensity was calculated relative to the reference sample.

### Results

ISG65 was successfully detected in all aliquots taken over a time course of 11 days (FIG. 15). After 11 days, in comparison to the reference sample taken immediately after addition of the protein into human plasma, 63% of the fluorescence intensity for ISG65 could still be detected.

The experiment shows a steady, but slow decline of the detected intensity over the measured time course towards the end of the experiment. The ISG65 aliquot taken at day 8 is considered to be an outlier. This can be accredited to the fact that the band already visually appears a lot smaller in comparison to the others, indicating a mistake in either sample preparation for the SDS-PAGE or when loading the samples onto the gel.

### Conclusion

This experiment confirms that ISG65 is stable over a prolonged period of time in human plasma ex vivo. Approximately, two thirds of the protein are still present after 11 days. No major fragmentation or shift in molecular weight of the detected ISG65 band could be observed, indicating that the remaining protein is likely functional.

### Functional Assessment of ISG65 and ISG65 M2 Activity in Human Plasma Over Time

After showing that ISG65 can be detected via Western Blot analysis for at least 11 days in human plasma in an ex vivo experiment, we set out to demonstrate that both ISG65 and ISG65 M2 retain their inhibitory function when incubated with human plasma over an extended period of time.

### Methods

Time course: 100 μL of ISG65 and 100 μL ISG65 M2 were added to 1350 μL of human plasma to a final concentration of approximately 1.5 mg/mL.

Reference samples were prepared by the addition of 100 μL PBS into 1350 μL of plasma. After thorough mixing, aliquots of the sample were taken immediately and flash frozen into liquid nitrogen (from hereon referred to as ‘Day 0’). All frozen aliquots were stored at −80° C. until further use. The sample tubes, closed and sealed with parafilm, were constantly incubated at 37° C., throughout the remainder of the experiment. Before removing aliquots, the sample tube was vortexed gently before briefly centrifuging the tube (<500×g) to collect the condensate from the lid. The appropriate sample volume was removed and diluted into PBS-T (1:20), before flash freezing in liquid nitrogen.

Haemolysis experiments: The plasma stability of ISG65 and ISG65 M2 were assessed using the commercially available AP50 test kit (HaemoScan, https://www.haemoscan.com/products/ap50/). In summary, the AP50 test exploits the fact that rabbit erythrocytes are lysed by human plasma or serum by action of the complement system. Lysis of the erythrocytes causes the release of haemoglobin, which subsequently can be determined via an absorbance measurement at 415 nm. A higher absorbance correlates with more lysis, caused by a higher activity of the complement. The AP50 test specifically asesses the activity of the alternative complement pathway.

The haemolysis experiment was performed according to the manufacturer's instructions (https://www.haemoscan.com/products/ap50/). In short, a dilution series, ranging from 2-fold to 15.2-fold dilution, was prepared for every aliqout taken throughout the time course experiment. For every timepoint, 4 samples were tested, plasma with and without ISG65 as well as plasma with and without ISG65 M2.

The absorbance measurements were performed in Nunc 96-well, transparent flat-bottom plates (ThermoScientific) and a Tecan Infinite 200 Pro microplate reader in conjunction with the Tecan Magellan software.

Determination of relative AP50: The terminology used within this section is coherent with the manual of the commercially available AP50 test kit. The term ‘AP50’ refers to the plasma fraction, the percentage of a tested sample in buffer, required to cause lysis of 50% of the rabbit erythrocytes. Therefore, a higher AP50 equates to lower complement activity (e.g. due to presence of an inhibitor). In our experiment, we have utilized the same measure of complement activity, however, instead of performing a lysis control with the lysis buffer provided by the kit to determine the maximum lysis, we have used the maximally achieved lysis by a sample on a given day. The remaining analysis and evaluation of the data were performed according to manufacturer's instructions.

### Results

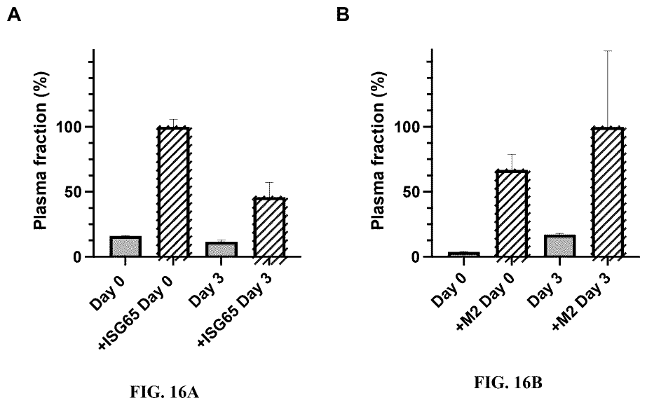

As expected, the complement activity of human plasma was severely inhibited on day 0, immediately after addition of ISG65 as an inhibitor. The AP50 of the human plasma with ISG65 added was 6-fold higher than the reference serum (100% vs 16%) (FIGS. 16A and 16B). Although the difference to the reference without added inhibitor was less pronounced, a significantly higher AP50 could also be observed for plasma with ISG65 on day 3.

We could show the same for ISG65 M2. The effect of the addition of M2 on day 0 was even more pronounced, causing 15-fold increase of the AP50 (FIGS. 16A and 16B). After 3 days the same significant difference to plasma without added inhibitor was still observabale. Within the error, the AP50 and therefore also the inhibitory effect, was also unchanged compared to day 0.

### Conclusions

These experiments underline the observations made in the plasma stability assessment using Western Blot analysis. ISG65 does not only appear stable in human plasma, but it does also appear to retain its capacity of binding to and therefore inhibiting the actions of human complement C3 and C3b over the time course observed in this experiment. The results obtained for ISG65 M2 indicate a higher stability in human plasma based on unchanged AP50 over time.

Human plasma when kept outside the human body at 37° C., does however age (e.g. break down of complement components over time, oxidation etc.). The results shown here can therefore not be directly compared to the results of the stability test of ISG65.

1. An ISG65-derived protein having the length of up to 500 amino acid residues and containing an amino acid sequence having at least 90% identity to the sequence SEQ ID NO: 8, wherein the underlined regions are conserved:
(SEQ ID NO: 8)
GYIEFELYRIDYWLEKLNGPKGRKDGYAKLSDSDIEKVKEIFNKAKDGI
TKQLPEAKKAGEEAGKLHTEVKKAAENARGQDLDDDTAKSTGLYRVLNW
YCITKEERHNATPNCDGIQFRKHYLSVNRSAIDCSSTSYEENYDWSANA
LQVALNSWEDVKPKKLESAGSDKNCNIGQSSESHPCTMTEEWQTPYKET
VEKLRELEDAYQRGKKAHDAMLGYANTAYAVNTKVEQE.

2. A method for administering ISG65-derived protein according to claim 1, for the treatment of a complement dysregulation disease.

3. A method for administering ISG65-derived protein according to claim 1, for the treatment of a disease selected from atypical hemolytic uremic syndrome (aHUS), C3 glomerulopathy (C3G), paroxysmal nocturnal hemoglobinuria (PNH), autoimmune diseases, Alzheimer's syndrome, schizophrenia, angioedema, macular degeneration, and Crohn's disease.

4. The ISG65-derived protein according to claim 1, wherein the ISG65-derived protein has the length of up to 500 amino acid residues and contains an amino acid sequence having at least 90% identity to the sequence SEQ ID NO: 5, wherein the underlined regions are conserved:
(SEQ ID NO: 5)
LLVIGSEDNRVPGDKKLTKEGAAALCKMKHLADKVAKERSQELKDRTQN
FAGYIEFELYRIDYWLEKLNGPKGRKDGYAKLSDSDIEKVKEIFNKAKD
GITKQLPEAKKAGEEAGKLHTEVKKAAENARGQDLDDDTAKSTGLYRVL
NWYCITKEERHNATPNCDGIQFRKHYLSVNRSAIDCSSTSYEENYDWSA
NALQVALNSWEDVKPKKLESAGSDKNCNIGQSSESHPCTMTEEWQTPYK
ETVEKLRELEDAYQRGKKAHDAMLGYANTAYAVNTKVEQEKPLTEVIAA
AKEAGKKGAKIIIPAAAPATPTNSTKNDDSAPTEHVDRGIATNETQVEV
GID.

5. The ISG65-derived protein according to claim 1, wherein the ISG65-derived protein has the length of up to 500 amino acid residues and contains an amino acid sequence having at least 90% identity to the sequence SEQ ID NO: 6, wherein the underlined regions are conserved:
(SEQ ID NO. 6)
MMKYLLVFAIIATRIPVLLVIGSEDNRVPGDKKLTKEGAAALCKMKHLA
DKVAKERSQELKDRTQNFAGYIEFELYRIDYWLEKLNGPKGRKDGYAKL
SDSDIEKVKEIFNKAKDGITKQLPEAKKAGEEAGKLHTEVKKAAENARG
QDLDDDTAKSTGLYRVLNWYCITKEERHNATPNCDGIQFRKHYLSVNRS
AIDCSSTSYEENYDWSANALQVALNSWEDVKPKKLESAGSDKNCNIGQS
SESHPCTMTEEWQTPYKETVEKLRELEDAYQRGKKAHDAMLGYANTAYA
VNTKVEQEKPLTEVIAAAKEAGKKGAKIIIPAAAPATPTNSTKNDDSAP
TEHVDRGIATNETQVEVGIDAD.

6. The ISG65-derived protein according to claim 1, wherein the ISG65-derived protein has the length of up to 500 amino acid residues and contains an amino acid sequence having at least 90% identity to the sequence SEQ ID NO: 7, wherein the underlined regions are conserved:
(SEQ ID NO: 7)
SEDNRVPGDKKLTKEGAAALCKMKHLADKVAKERSQELKDRTQNFAGYI
EFELYRIDYWLEKLNGPKGRKDGYAKLSDSDIEKVKEIFNKAKDGITKQ
LPEAKKAGEEAGKLHTEVKKAAENARGQDLDDDTAKSTGLYRVLNWYCI
TKEERHNATPNCDGIQFRKHYLSVNRSAIDCSSTSYEENYDWSANALQV
ALNSWEDVKPKKLESAGSDKNCNIGQSSESHPCTMTEEWQTPYKETVEK
LRELEDAYQRGKKAHDAMLGYANTAYAVNTKVEQEKPLTEV.

7. The ISG65-derived protein having the length of up to 500 amino acid residues and containing an amino acid sequence having at least 98% identity to the sequence SEQ ID NO: 12, wherein the underlined and bolded-underlined amino acids are conserved:
(SEQ ID NO: 12)
YIEFELYRIDYWLEKLNGPKGRKDGYAKLSDSDIKKVKEIFEKAKDGIT
KQLPEAKKAAEEAEKLHQEVKEAAEKARGQDLDDDTAKSTGLYRVLNWY
CITKEERHNATPNCDGIQFRKHYLSVNRSAIDCSSTSYEENYDWSANAL
QVALNSWEDKKPKKLESAGSDKNCNIGQSSESHPCTMTEEWQTHYKETI
EKLRELEEAYQRGKKAHDDMLGYANTAYAVNTKVEQEKPLS.

8. The ISG65-derived protein according to claim 7, having the length of up to 500 amino acid residues and containing an amino acid sequence having at least 98% identity, to the sequence SEQ ID NO: 14, wherein the underlined and bolded-underlined amino acids are conserved:
(SEQ ID NO: 14)
SEDNRVPGDKKLTKEGAAALCKMKHLADKVAKERSQELKDRTQNFAGYI
EFELYRIDYWLEKLNGPKGRKDGYAKLSDSDIKKVKEIFEKAKDGITKQ
LPEAKKAAEEAEKLHQEVKEAAEKARGQDLDDDTAKSTGLYRVLNWYCI
TKEERHNATPNCDGIQFRKHYLSVNRSAIDCSSTSYEENYDWSANALQV
ALNSWEDKKPKKLESAGSDKNCNIGQSSESHPCTMTEEWQTHYKETIEK
LRELEEAYQRGKKAHDDMLGYANTAYAVNTKVEQEKPLSEV.

9. The ISG65-derived protein according to claim 7, having the amino acid sequence:
(SEQ ID NO: 14)
SEDNRVPGDKKLTKEGAAALCKMKHLADKVAKERSQELKDRTQNFAGYI
EFELYRIDYWLEKLNGPKGRKDGYAKLSDSDIKKVKEIFEKAKDGITKQ
LPEAKKAAEEAEKLHQEVKEAAEKARGQDLDDDTAKSTGLYRVLNWYCI
TKEERHNATPNCDGIQFRKHYLSVNRSAIDCSSTSYEENYDWSANALQV
ALNSWEDKKPKKLESAGSDKNCNIGQSSESHPCTMTEEWQTHYKETIEK
LRELEEAYQRGKKAHDDMLGYANTAYAVNTKVEQEKPLSEV.

10. The ISG65-derived protein according to claim 7, having the length of up to 500 amino acid residues and containing an amino acid sequence having at least 98% identity, to the sequence SEQ ID NO: 13, wherein the bolded regions and the underlined amino acids are conserved:
(SEQ ID NO: 13)
MMKYLLVFAIIATRIPVLLVIGSEDNRVPGDKKLTKEGAAALCKMKHLA
DKVAKERSQELKDRTQNFAGYIEFELYRIDYWLEKLNGPKGRKDGYAKL
SDSDIKKVKEIFEKAKDGITKQLPEAKKAAEEAEKLHQEVKEAAEKARG
QDLDDDTAKSTGLYRVLNWYCITKEERHNATPNCDGIQFRKHYLSVNRS
AIDCSSTSYEENYDWSANALQVALNSWEDKKPKKLESAGSDKNCNIGQS
SESHPCTMTEEWQTHYKETIEKLRELEEAYQRGKKAHDDMLGYANTAYA
VNTKVEQEKPLSEVIAAAKEAGKKGAKIIIPAAAPATPTNSTKNDDSAP
TEHVDRGIATNETQVEVGIDAD.

11. The ISG65-derived protein according to claim 7, having the amino acid sequence:
(SEQ ID NO: 13)
MMKYLLVFAIIATRIPVLLVIGSEDNRVPGDKKLTKEGAAALCKMKHLA
DKVAKERSQELKDRTQNFAGYIEFELYRIDYWLEKLNGPKGRKDGYAKL
SDSDIKKVKEIFEKAKDGITKQLPEAKKAAEEAEKLHQEVKEAAEKARG
QDLDDDTAKSTGLYRVLNWYCITKEERHNATPNCDGIQFRKHYLSVNRS
AIDCSSTSYEENYDWSANALQVALNSWEDKKPKKLESAGSDKNCNIGQS
SESHPCTMTEEWQTHYKETIEKLRELEEAYQRGKKAHDDMLGYANTAYA
VNTKVEQEKPLSEVIAAAKEAGKKGAKIIIPAAAPATPTNSTKNDDSAP
TEHVDRGIATNETQVEVGIDAD.

12. The ISG65-derived protein according to claim 7, having the length of up to 500 amino acid residues and containing an amino acid sequence having at least 98% identity, to the sequence SEQ ID NO: 11, wherein the bolded regions and the underlined amino acids are conserved:
(SEQ ID NO: 11)
LLVIGSEDNRVPGDKKLTKEGAAALCKMKHLADKVAKERSQELKDRTQN
FAGYIEFELYRIDYWLEKLNGPKGRKDGYAKLSDSDIKKVKEIFEKAKD
GITKQLPEAKKAAEEAEKLHQEVKEAAEKARGQDLDDDTAKSTGLYRVL
NWYCITKEERHNATPNCDGIQFRKHYLSVNRSAIDCSSTSYEENYDWSA
NALQVALNSWEDKKPKKLESAGSDKNCNIGQSSESHPCTMTEEWQTHYK
ETIEKLRELEEAYQRGKKAHDDMLGYANTAYAVNTKVEQEKPLSEVIAA
AKEAGKKGAKIIIPAAAPATPTNSTKNDDSAPTEHVDRGIATNETQVEV
GID.

13. The ISG65-derived protein according to claim 7, having the amino acid sequence:
(SEQ ID NO: 11)
LLVIGSEDNRVPGDKKLTKEGAAALCKMKHLADKVAKERSQELKDRTQN
FAGYIEFELYRIDYWLEKLNGPKGRKDGYAKLSDSDIKKVKEIFEKAKD
GITKQLPEAKKAAEEAEKLHQEVKEAAEKARGQDLDDDTAKSTGLYRVL
NWYCITKEERHNATPNCDGIQFRKHYLSVNRSAIDCSSTSYEENYDWSA
NALQVALNSWEDKKPKKLESAGSDKNCNIGQSSESHPCTMTEEWQTHYK
ETIEKLRELEEAYQRGKKAHDDMLGYANTAYAVNTKVEQEKPLSEVIAA
AKEAGKKGAKIIIPAAAPATPTNSTKNDDSAPTEHVDRGIATNETQVEV
GID.

14. A method of administering ISG65-derived protein according to claim 7, as treatment to a subject in need thereof.

15. A method of administering ISG65-derived protein according to claim 7, in the treatment of a complement dysregulation disease.

16. A method of administering ISG65-derived protein according to claim 7, in the treatment of a disease selected from atypical hemolytic uremic syndrome (aHUS), C3 glomerulopathy (C3G), paroxysmal nocturnal hemoglobinuria (PNH), autoimmune diseases, Alzheimer's syndrome, schizophrenia, angioedema, macular degeneration, and Crohn's disease.
